(19) United States
(12) Patent Application Publication
(10) Pub. No.: US 2011/0306853 A1
(43) Pub. Date: Dec. 15, 2011

(54) Body fluid sampling/fluid delivery device

(76) Inventors: Michael Darryl Black, Palo Alto, CA (US); Anita Margarette Chambers, Goleta, CA (US); Richard Chambers, Evans, GA (US); Nihat Okulan, Los Angeles, CA (US)

(21) Appl. No.: 13/052,887

(22) Filed: Mar. 21, 2011

Related U.S. Application Data
(60) Provisional application No. 61/315,736, filed on Mar. 19, 2010.

(51) Int. Cl. A61B 5/157; A61M 5/00; A61B 5/15

(52) U.S. Cl. 600/309; 600/575; 604/173

(57) ABSTRACT

A body fluid sampling or fluid delivery system includes a polymeric support and an array of polymeric microneedles coupled to the support, each of a microneedle having a height of 500 to 2000 μm and a tapering angle of 60 to 90°. A plurality of polymeric microchannels are provided with being associated with a microneedle. The plurality of polymeric microchannels are integrally formed with the array of polymeric microneedles without bonding. At least one polymeric reservoir is coupled to the plurality of microchannels.

### BACKGROUND OF THE INVENTION

1. Field of the Invention

This invention relates generally to body fluid sampling/fluid delivery devices, and more particularly to body fluid sampling/fluid delivery devices, their methods of use and manufacture, that is suitable for neonates, children and adult humans as well as juvenile and adult animals and does not induce unnecessary trauma to the patient. This invention also relates to a monolithically integrated device that is constructed by a single type of polymer. Monolithic (vs. hybrid) integration is an integration of two functional components with minimum/zero change in either the performance or the manufacturing process of each.

2. Description of the Related Art

Although advances in biomedical technology and novel therapies have allowed for a significant decrease in neonatal mortality, the same cannot be said in regards to neurodevelopment morbidity. For the critically ill newborn, the first week of life is a source of repeated and uncontrollable noxious events, which, to date have poorly understood long-term consequences. Just like their adult counterparts, sick neonates require a multitude of daily blood tests to diagnose and monitor their health status and the effectiveness of therapies they are undergoing. However unlike their adult counterparts, blood collection for laboratory testing is the predominate source of non-physiologic anemia of prematurity (AOP) in both Low Birth Weight (VLBW<1,500 gm) and Very Low Birth Weight (ELBW<1,000 gm) and Extremely Low Birth Weight (ELBW<500 gm) neonates.

Even ELBW neonates experience pain, an understanding that was not well accepted just 20 years ago. At that time, neonates did not receive analgesia even while undergoing surgery. It is probable that repetitive daily-uncontrolled trauma may in part explain noted behavioral difficulties in ex-premature children. The standard of care for collecting blood samples in neonates today is “the heel prick.” It requires using a sharp lance, which is penetrated into the heel of the infant while the ankle is firmly restrained. The foot is vigorously squeezed to force enough blood from the injured heel to perform the laboratory tests. As long as the child remains critically ill, the heel prick is required every 4 to 6 hours. Over a period of one week approximately one-half of the neonates' total circulating blood is removed from the body.

The heel prick is the preferred method of obtaining capillary blood samples when either a direct invasive line into an artery is unavailable or when the mandated newborn baby screen is required. State legislation and protocols have been written in an attempt to standardize this “patient friendly” heel prick technique, but evidence suggests that even intra-venous morphine and/or local anesthesia remains insufficient in preventing the intense pain encountered by the sick infant with this heel prick technique.

Besides the standard techniques in alleviating the stress response and pain associated with procedures such as the heel prick, pharmacological techniques i.e. sedation, paralysis and opiates, a number of newer non-pharmacological techniques are taking root. “Sensorial saturation”, a technique combining visual, tactile and auditory stimulation to overwhelm the sense, has been shown to limit the physical response seen in association with the heel prick, but one can not assume that the pain and trauma have been eliminated merely because the sense are overwhelmed. Additionally, the Newborn Individualized Developmental Care and Assessment Program (NIDCAP) offers the promise of improving short-term respiratory and long-term behavioral and developmental when performed in combination with the stressful event. However, in all of these cases, one cannot assume that because the sick infants physical response is reduced, there is a corresponding reduction in the pain and trauma experienced.

Preterm critically ill newborns are among the most heavily transfused patient cohorts. All preterm infants experience a postnatal decrease in hemoglobin levels. A myriad of processes are responsible for Anemia of Prematurity (AOP), some of which are expected, i.e. developmentally regulated physiologic processes, while others remain pathologic and iatrogenic. Unlike more mature infants, the premature neonates, Low Birth Weight (LVW) infants, Very Low Birth Weight (BLBW) infants and Extremely Low Birth Weight (ELBW) infants frequently become clinically symptomatic to all sources of AOP thus mandating transfusion. In order to assess health or treatment effectiveness it is common for critically ill LBW, VLBW and ELBW infants to have nearly one-half of their total circulating blood supply removed (primarily with the heel prick method) every week during hospitalization for laboratory testing. Since, red blood cell (RBC) transfusion remains the mainstay of therapy for AOP, an estimated 2.7 million of such procedures was performed last year in the United States.

There is a vast gap between the capability of emerging technology to be utilized in developing miniaturized devices to more humanely diagnose and treat these critically ill premature, LBW, VLBW and ELBW infants and what is considered “standard of care” today. Due to the immaturity of these infants, they are forced to undergo procedures that cause significant pain, trauma, and even medically induced anemia leading to subsequent blood transfusions, which cause even further pain and trauma. It is a vicious cycle of noxious stimuli to force anyone, especially an immature infant to endure. From a clinical perspective, although this treatment is inhuman, there is no other way because the medical technology does not exist to eliminate these noxious stimuli. From a scientific perspective, nanotechnology could be utilized to develop a device specifically for these infants that would vastly reduce the amount of noxious stimuli and the subsequent downstream complications such as possible infection and psychological and physiological trauma. The technology is available, but it has not been adapted to meet the needs of these at risk children. This exemplifies the gap between science for the sake of science and science for the sake of helping improve the quality of life and the health of patients, in this case, pediatric patients who do not have the emotional or mental capability to understand why they are being made to suffer.

Accordingly, there is a need to diagnose and treat premature, LBW, VLBW and ELBS infants with reduced or no pain.

Although neonates and infants represent the extreme in terms of the importance of a painless, atraumatic method of removing fluids from, or inserting fluids into the body, it is also important to all human beings. While there is little risk of blood drawing requiring subsequent blood transfusions in adults, the issue of pain and trauma does still exist as it does in the neonate,especially in adults forced to undergo daily or multiple daily blood drawing, fluid delivery, pharmacologic delivery, and the like.

In veterinary use, the same three issues exist. In all animals, a painless, atraumatic method of removing fluids from, or inserting fluids into the body is important. In a way, animals are similar to neonates in that they do not understand the pain and trauma associated with clinical activities and have no means by which to avoid these noxious stimuli. Large animals such as horses, mules, donkeys, cows, etc. are more similar to human adults in that the risk of required blood transfusions from drawing blood is low. However, with many small animals such as birds, rodents, reptiles, dogs, cats, etc. are more similar to the situation with neonates as described in the section above.

### SUMMARY OF THE INVENTION

An object of the present invention is to provide an improved body fluid sampling/fluid delivery device.

Another object of the present invention is to provide methods and fabrication processes for creating an improved body fluid sampling/fluid delivery device.

A further object of the present invention is to provide polymer microneedles for a body fluid sampling/fluid delivery device.

Yet another object of the present invention is to provide a body fluid sampling/fluid delivery device with integrated microneedles and microfluidics.

Another object of the, present invention is to provide a body fluid sampling/fluid delivery device that collects low volumes of body fluids with little or no pain.

A further object of the present invention is to provide a body fluid sampling/fluid delivery device suitable for neonates that does not induce unnecessary trauma to the human or animal patients including neonatal, child and adult humans and large and small animals.

Still another object of the present invention is to provide a body fluid sampling/fluid delivery device that is suitable for performing blood gas concentration analysis every 4-6 hours on hospitalized neonates.

Still another object of the present invention is to provide a body fluid sampling/fluid delivery device that is suitable for performing diagnostic analysis (included but not limited to pharmacological testing, hematological analysis, body fluid analysis including but not limited to lymphatic fluid, interstitial fluid, urine, cerebrospinal fluid, intraocular fluids, biliary and ductal fluids, and intra-cellular fluids); therapeutic treatments (including but not limited to the delivery of pharmaceuticals, vaccinations, vitamins, minerals, therapeutic supplements, and the like); genomic diagnostics and gene removal (including but not limited to the analysis of genetic diseases and disorders, stem cell removal, genetic material removal, and the like); and genetic therapies (including but not limited to the delivery of stem cells, the delivery of genetic materials into intraocular fluid, the delivery of genetic materials into intracellular spaces, and the like).

Still another object of the present invention is to provide a body fluid sampling/fluid delivery device that is suitable for performing general blood work.

Another object of the present invention is to provide a body fluid sampling/fluid delivery device that improves the method of drawing blood from neonates without a heel prick.

A further object of the present invention is to provide a body fluid sampling/fluid delivery device that performs body fluid sample analysis inside a patch, 12 allowing for more accurate results compared to subjecting the body fluid to room air contaminating which can cause the O₂ analysis to be inaccurate.

Yet another object of the present invention is to provide a body fluid sampling/fluid delivery device that requires only 1-2 drops of blood.

Yet a further object of the present invention is to provide a monolithically (one polymer, no bonding) integrated method for manufacturing microneedles with microfluidic devices.

These and other objects of the present invention are achieved in, A body fluid sampling or fluid delivery system that includes a polymeric support and an array of polymeric microneedles coupled to the support, each of a microneedle having a height of 500 to 2000 μm and a tapering angle of 60 to 90°. A plurality of polymeric microchannels are provided with being associated with a microneedle. The plurality of polymeric microchannels are integrally formed with the array of polymeric microneedles without bonding. At least one polymeric reservoir is coupled to the plurality of microchannels.

In another embodiment of the present invention, a method is provide for sampling a body fluid from a patient. A system is provided with an array of microneedles. AT least a portion of the system is integrally formed. The array of microneedles are introduced into a patient. A body fluid is collected from the patient in the sample chamber. A parameter of the body fluid in the sample chamber is measured.

### BRIEF DESCRIPTION OF THE DRAWINGS

FIGS. 1a-d illustrate the penetration of microjets into gel and human skin in vitro.

FIG. 2a is an illustration of one embodiment of a body fluid sampling/fluid delivery system of the present invention.

FIG. 2b is a schematic of a pulsed microjet device in one embodiment of the present invention.

FIG. 3 is a micrograph showing silicon microneedles

FIG. 4 is the cad layout of a microneedle punch.

FIG. 5 is a schematic showing the microneedle array inserted into skin to draw capillary blood.

FIG. 6 is a cross-section of a reservoir in one embodiment of the present invention.

FIG. 7 is a schematic of the microneedle array.

FIG. 8 is the microneedle type structure using reactive ion etch.

FIG. 9 shows a polymide wafer (patch).

FIG. 10 depicts the fabrication steps of the microneedle layer and sensing layer, with both layers bonded to form channels and a reservoir.

FIG. 11a-b are graph of the volume of each microjet and the amount of liquid ejected.

FIG. 12 depicts the penetration of microjets into human skin in vitro, showing the intact structure of corneocyttes around the injection site.

FIG. 13a-b are graphs of the volume of jet delivered across the epidermis, and relative blood glucose levels

FIG. 14 shows the operational principal of the sensor inside the microchannel.

FIG. 15 illustrates an embodiment of a controllable force driver in the form of a flat electric lancet driver that has a solenoid-type configuration.

FIG. 16 illustrates an embodiment of a controllable force driver in the form of a cylindrical electric lancet driver using a coiled solenoid -type configuration.

FIG. 17 illustrates a displacement over time profile of a lancet driven by a harmonic spring/mass system.

FIG. 18 illustrates the velocity over time profile of a lancet driver by a harmonic spring/mass system.

FIG. 19 illustrates a displacement over time profile of an embodiment of a controllable force driver.

FIGS. 20 illustrates a velocity over time profile of an embodiment of a controllable force driver.

FIG. 21 illustrates the lancet microneedle partially retracted, after severing blood vessels; blood is shown following the microneedle in the wound tract.

FIG. 22 illustrates blood following the lancet microneedle to the skin surface, maintaining an open wound tract.

FIG. 23 shows an embodiment according to the present invention of a system for providing remote analysis of medical data.

FIG. 24 shows an embodiment of the method according to the present invention.

FIG. 25 embodiment of a medical device medical data record.

FIGS. 26 through 34 illustrate a method of making the body fluid sampling/fluid delivery system of the present invention.

### DETAILED DESCRIPTION OF THE PREFERRED EMBODIMENTS

In various embodiments, the present invention is a body fluid sampling/fluid delivery system. Methods and fabrication processes for the body fluid sampling/fluid delivery system are provided as are, polymer microneedles, polymer microfluidic systems, and the integration of a microneedle with a microfluidic system.

In one specific embodiment, the present invention is a body fluid sampling/fluid delivery system that uses a patch, also known as a substrate, which can be nanotechnology based, to sample blood painlessly, without trauma, and without causing anemia. This embodiment is particularly useful for premature infants, but can also be used for older children and adults. As a non-limiting example, the body fluid sampling/fluid delivery system of the present invention, reduces or eliminates the traumatic heel prick method of blood collection in neonates, more particularly, (i) trauma leading to neurological deficits, (ii) iatrogenic anemia leading to blood transfusions, and (iii) inaccuracy of analyzing room air contaminated blood samples. As a non-limiting example, the body fluid sampling/fluid delivery system provides a more humane method of drawing blood from premature infants, reduces the health risks and costs associated with experiencing undue trauma and blood transfusions, and does so while providing more accurate blood analysis results.

In one specific embodiment, the body fluid sampling/fluid delivery system 10 can be used for neonate, LBW, VLBW or ELBW infants. As a non-limiting example, a polymer blood sampling patch, can be used. Suitable sampling patch materials can include silicon, polymers and metal substrates, which lay the groundwork for an immediate digital record which matches the patient's unique blood data with the patient's unique medical number, mitigating errors associated with improper patient identification. Electronics can be included in a patch for electronic processing and receipt of patient data. In one embodiment, the present invention uses microneedles.

A microneedle is a needle-shaped device used in biological and medical applications. It serves as a tool/microchannel 16 to conduct liquids in (drug delivery) and out of (extraction of blood and/or other bodily fluids) the skin. The microscopic dimensions (typical range: length: tens of microns to 1-2 millimeters; tip diameter: fraction of a micron to tens of microns) diminish the physical impact on bodies (humans and animals), thus reducing pain. Its manufacturing process often facilitates the integration to micro- and nano-fluidics, which provides sensitive detection of biomedical signals such as blood gas. Such integration reduces the total amount of liquids involved, increases detection accuracy, and (significantly) trims down cost.

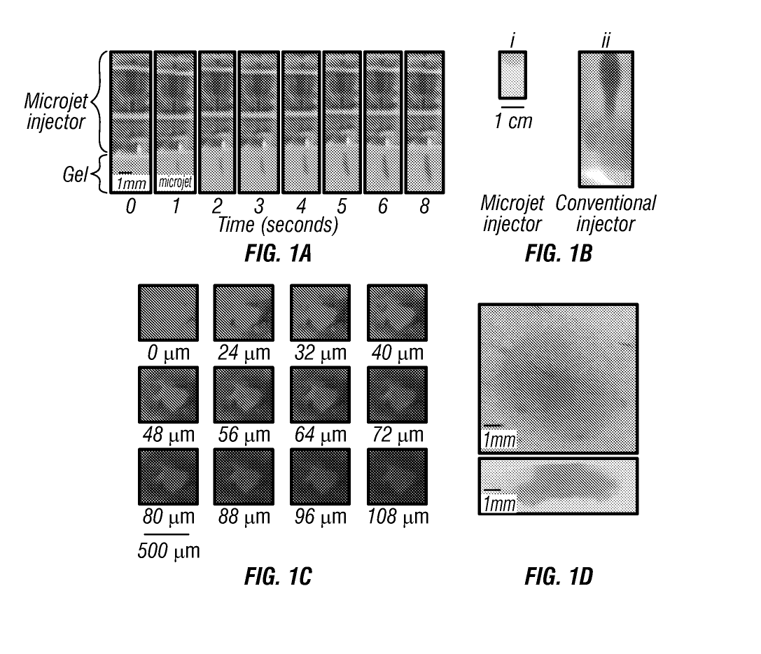

FIGS. 1a-d illustrate the penetration of microjets, e.g., microneedles, into gel and human skin in vitro.

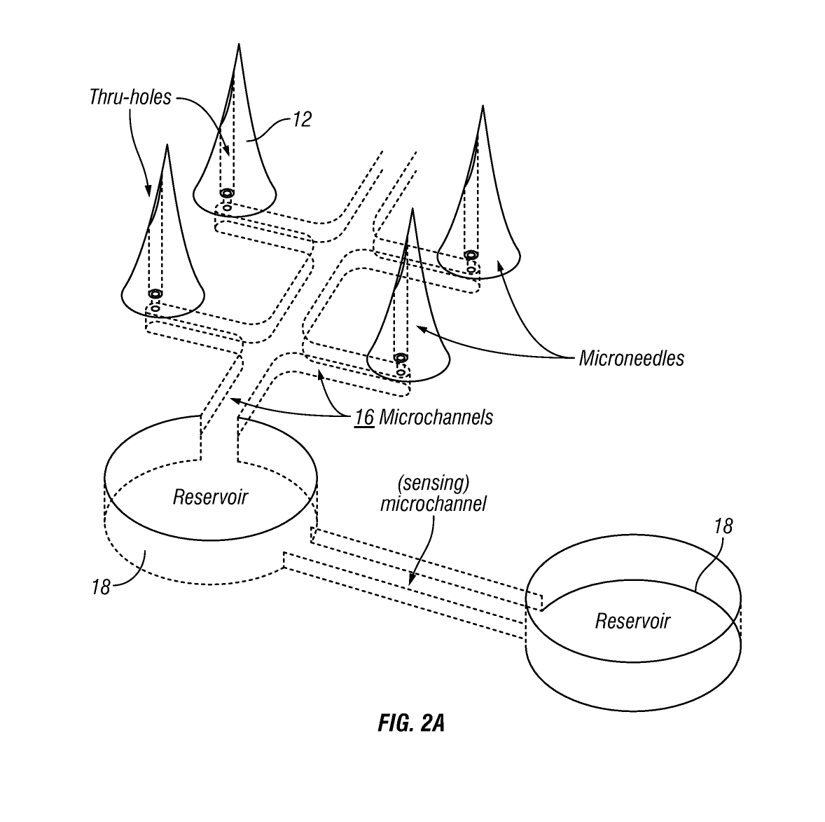

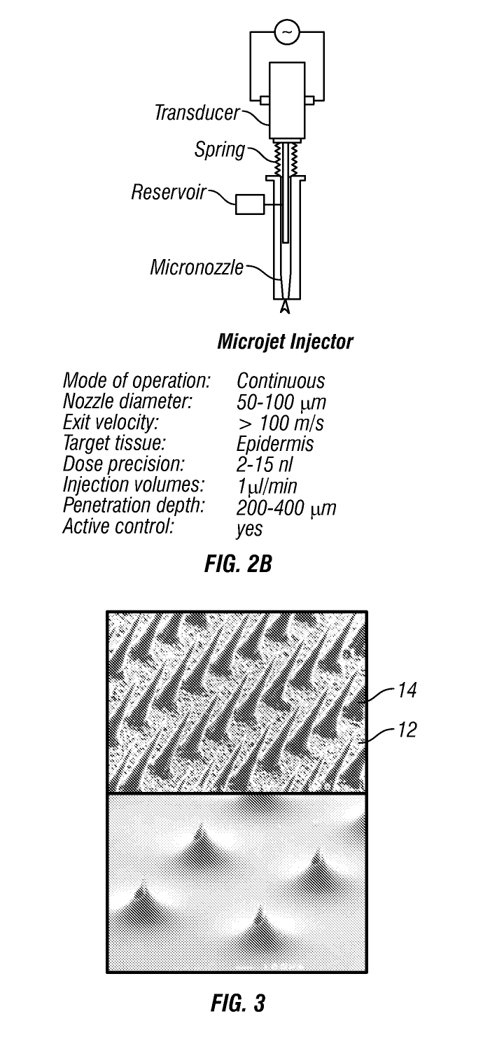

Referring now to FIG. 2(a) the body fluid sampling or fluid delivery system 10 includes, a polymeric support 12, an array of microneedles 14 coupled to the support 12. In one embodiment, the microneedles 14 have a height of 500 to 2000 μm and a tapering angle of 60 to 90°. A plurality of polymeric microchannels 16 are provided, each of a microchannel 16 being is associated a microneedle 14. The plurality of polymeric microchannels 16 are integrally formed with the array of polymeric microneedles 14 without bonding and are integrated as one. At least one polymeric reservoir 18 is coupled to the plurality of microchannels 16. In one embodiment, the polymeric support 12 is coupled to the array of polymeric microneedles without external bonding. The plurality of polymeric microchannels 16 and the array of microneedles 14 are integrally formed to provide for controlled dimensions and alignment of the microchannels 16 with the microneedles 14. In one embodiment, the support 12, microneedles 14, microchannels 16 and the reservoir 18 are formed of the same polymer and are all integrally formed.

The analysis of a body fluid substance can be in the microchannels 16 or the reservoir 18. In one embodiment, first and second reservoirs 18 are provided for incoming and outgoing fluids. It will be appreciated that any number of reservoirs 18 can be included. The microchannels 16 can be capillary channels which do not provide for a back pressure for pull. In one embodiment, the size of the reservoir 18 or reservoirs 18 in total is no great than 1 μL.

As illustrated in FIG. 2(b), the present invention is a body fluid sampling/fluid delivery system 10 is configured to provide withdrawal of a body fluid, including but not limited to blood, a blood gas, and the like, and can also be utilized to inject a fluidic medium, as more fully explained hereafter.

In one embodiment, the body fluid sampling/fluid delivery system 10 is a monolithically formed, e.g., with no bonding involved, multi-layer polymer microfluidic system. In one embodiment the polymer is SU-8 which provides structures with large out-of-plane dimensions. SU-8 is a good structural polymer because of its unique optical properties under UV (minimum absorption for wavelengths greater than 365 μm after exposure-caused cross-linking), which enables the process capability of producing high aspect ratio microstructures (that follow the contour of the incoming exposure).

It will be appreciated that other polymers can be used that may require different subsequent processing techniques. As a non-limiting example, polyimide offers similar mechanical strength, but requires dry etching to create a tapering-shaped microneedle 14 and bonding for the integration of microfluidics. Other suitable polymers include but are not limited to PMMA, PMGI, BCB, and the like.

The microneedles 14 can be have an off-centered through hole for blood transport. Microneedle 14 taper control, which can provide optimal penetration with limited material hardness, can be achieved via placement of an UV mask material Plasma sharpening can be used to sharpen the microneedles 14, particularly polymeric microneedles 14. A subsequent material deposition for improved modulus and hardness can be provided. The deposited materials enhance the hardness of the polymer and can include metals such as titanium, nickel, tungsten, and the like; dielectrics such as silicon oxide, silicon nitride and the like. A higher modulus is desired since the microneedle's mechanical strength, or resistance to lateral bending force, is strongly dependent on (˜to the cubic power of) it. In one embodiment, when SU-8 is the polymer it has a modulus of SU-8 of about 2-5 GPa and is one of the highest among polymers, it is still far below that of metals and dielectrics (typically ˜50-200 GPa). The thickness of coating material is determined primarily by process compatibility, such as CTE mismatch, interface adhesion, and the like. In one embodiment, the range about 1-10 um.

In one embodiment, tapered polymeric structures are created by, (i) overexposure, (ii) near-field diffraction, (iii) mask distance adjustment, (iv) using external micro-lenses or diffuser lithography to change the incident angle of the UV, and the like. Tapers in the polymeric structures offer flexible structural topologies. Another technique that can cause the change of incidence angel is diffuser lithography. For polymeric microneedles 14 a taper can significantly improve the success rate of microneedle 14 insertion due to the limited strength. There are many ways to produce tapers including but not limited to, (i) overexposure (light scattering and slight change of absorption after exposure lead to exposure of the polymer or any light-sensitive polymer-beyond direct line-of-sight), and (ii) near-field diffraction and mask distance adjustment (which in one process allows the placement of an UV mask at different distances from the polymer, thus producing diffraction effects which result in change of exposure profile).

Multi-wavelength exposure provides absorption increases as the wavelength drops from 365 nm, thus enabling fabrication of three dimensional depth dependent structures such as microneedles 14 and microfluidics such as the microchannel 16 s.

In one embodiment, the body fluid sampling/fluid delivery system 10 of the present invention includes a microneedle 14 or an array of microneedles 14 coupled to a support member or patch 12, a micro-fluidics system, a micro-injector and one or more displays. In another embodiment, the microneedle 14 or microneedle array 14 is replaced with a microjet or other suitable mechanisms, as more fully discussed hereafter. Micro-biosensors can be coupled to the patch 12. As a non-limiting example, the patch 12 can be 5 mm by 10 mm.

As non-limiting examples, (i) the microneedle 14 height can be 500 to 2000 μm, (ii) a tapering angle, in degrees, for the microneedles 14 is 90 to 60, (iii) microneedle 14 pitch is 400 to 2000 μm, (iv) a patch 12 dimension is 5 to 10 mm (squared) and (v) the number of microneedles 14 per patch 12 is 9 to 250.

FIG. 2(b) Illustrates Microjet Injectors of the Present Invention.

Referring now to FIG. 3, the microneedle array 14 is more fully illustrated. The use of an array of microneedles 14 provides a minimally invasive method to transfer molecules into and out of skin. The small size and extremely sharp tips minimizes or eliminates the tissue trauma and insertion pain experienced by the patient. The length of the microneedles 14 can be specifically designed to avoid penetration into the pain receptors inside the inner layers of the skin to draw capillary blood samples. Additionally, the openings of the hollow microneedles 14 can be made large enough to enable a relatively high rate of blood sample withdrawal or drug delivery.

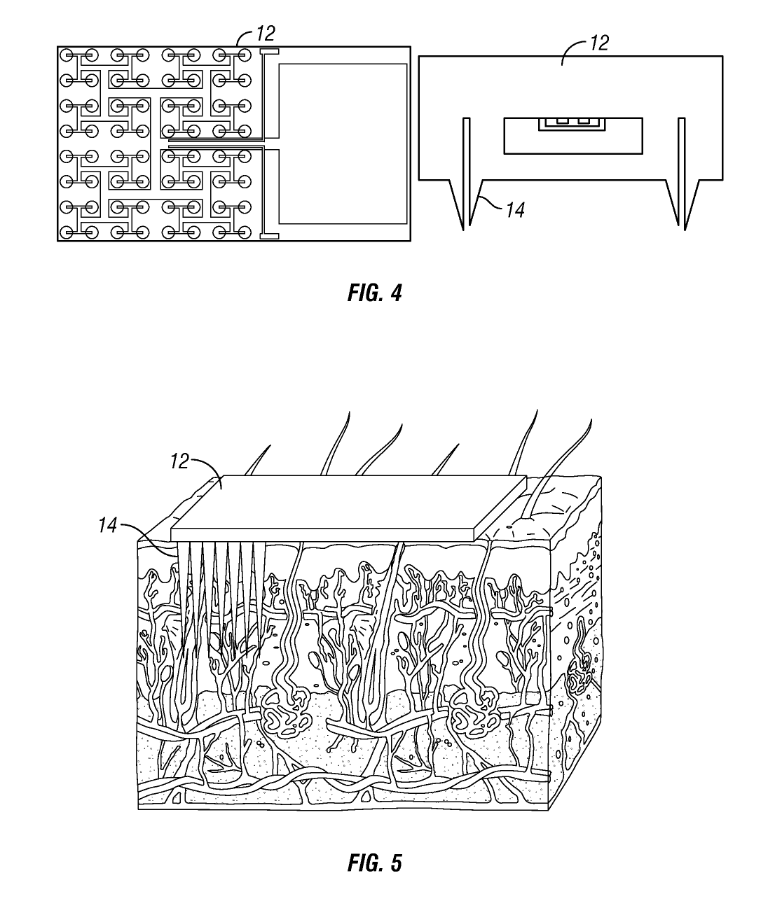

As a non-limiting example, FIG. 4 illustrates an embodiment of a microneedle patch 12 of the present invention. The left image of FIG. 4 shows a CAD layout of the microneedle patch 12. After the patch 12 is inserted into the skin, blood flows through the microneedle channels and into the reservoir 18. In one embodiment, the microchannels 16 are designed in a way such that each channel path, from the microneedle 14 until the back pressure reservoir, sees the same flow resistance. As a non-limiting example, less than 1 μL is used to fill all the microchannels 16 and the reservoir 18. The left image of FIG. 4 shows the cross-sectional view of the sensing chamber and of two adjacent microneedles 14.

As a non-limiting example, the patch 12 can be 5 mm by 1 mm in size and includes microneedles 14. The fabrication of multiple microneedles 14 can be achieved on a wafer level, similar to the fabrication of IC chips.

The left image of FIG. 4 shows the cross-sectional view of the sensing chamber and of two adjacent microneedles 14.

The left image of FIG. 4 shows the cross-sectional view of the reservoir 18 and of two adjacent microneedles 14.

FIG. 5 illustrates the microneedle array 14 of the present invention positioned to draw blood without being in contact with pain receptors.

In one embodiment, when the microneedles 14 are hollow, the microneedles 14 are sized to be small enough to draw only interstitial fluid and large enough to draw whole blood. If a microneedle 14 is not hollow, then it's tip dimension is as small as possible subject to manufacturing limitations, and can be 300 um to 1 um. As a non-limiting example, the dimension of a microneedle tip at the narrowest point of the tip can be in the range of 1 nm to 300 um. The largest cell in whole blood is a monocyte which typically has a width of about 10-30 um. 300 um allows 10 monocytes to travel through the microneedle tip simultaneously.

The length of the microneedles 14 can vary. In one embodiment the length of the microneedles 14 can be selected be in the range short sufficient to draw only interstitial fluid and long enough to draw venous blood. As a non-limiting example, the microneedle 14 length can be in the range of 100 um-2.0 cm. The diameter of the microneedle 14 (OD) can be 20-gauge (1 mm) to 20 um. The lumen or hole can be 1 um to 1 mm. The microchannels 16 can be 1 um to 3 mm. The injector nozzle can be 0.9 mm to 1 um. The injector can inject 2 um to 2 centimeters (typical dimensons of microchannels: length: 0.5 um to 5 cm; width: 10 um to 500 um; height: 1 um to 500 um).

The microneedles 14 can be in a variety of different shapes. In one embodiment, the shape of the microneedle 14 is selected for the type of fluid that is either collected from or injected into the patient. As non-limiting examples, suitable microneedle 14 shapes include but are not limited to, cylindrical, semi-cylindrical, conical, flat-sided, step pyramidal, a combination of different distal tip geometries, straight, diagonal, angled, and the like.

In various embodiments, the microneedles 14 can be hollow or solid. When the microneedles 14 are solid, a penetration is made through the skin surface and fluid flows around the microneedle 14. In this embodiment, the microneedle 14 remains at the selected tissue site for a sufficient time for fluid to flow preferably unaided by vacuum, and the like. Spontaneous flow is desired. With a hollow microneedle 14, the hollow orifice can be at any location of the microneedle 14. In one embodiment, the orifice is offset and not in the center of the distal portion, which can be, by way of example, a conical geometry.

The body fluid sampling/fluid delivery system 10 does not require the application of a vacuum through or around a microneedle 14 for the withdrawal of body fluid. Instead, the body fluid sampling/fluid delivery system 10 can utilize backpressure to body fluid flow, such as that provided by capillary action provided by the microchannels 16 of the body fluid sampling/fluid delivery system 10. If a vacuum is used, it can be in the range of 10⁻³ to 750 mmHg. In one embodiment where the microneedle 14 is hollow, the distal penetrating end of the orifice can be open and uncovered, or may include a protective cover over the tip to prevent clogging. The protection cover can be a cap type of member positioned at the distal end of the microneedle 14. In another embodiment, a seal is provided that is not in contact with the distal end of the microneedle 14. The seal can be broken when the distal is launched by the distal end of the microneedle 14, or a seal breaker can be provided. Additionally, when the microneedle 14 is hollow, the orifice can be single or multiple. The multiple dimensions can be utilized to filter the whole blood, separating out the plasma for analysis. To protect the sample of blood from ambient air contamination using a non-hollow microneedle 14, a diaphragm can be used and made from polymer.

With a plurality or array of microneedles 14, the dimensions between adjacent microneedles 14 can vary. As a non-limiting example, the distance between microneedles 14 in the array can be about 2 um to 5 mm.

The amount of force or pressure requirement to apply to the patch 12 can vary. As a non-limiting example, the amount of force can be in the range of about 0.01 to 10 Newtons of force to penetrate the skin. In other embodiments, additional force of the entire arm can be instead of a single finger.

The microneedle 14 array can include any desired number of microneedles 14, including but not limited to 1 to 1 million. A preferred number of microneedles 14 can be 1 to 100,000 microneedles 14. As a non-limiting example, the microneedle array 14 can have a total area (height×width) of 1 μm² to 1 cm². This dimension of microneedle array 14 is particularly useful for injecting mesotherapy compounds.

It will be appreciated that the shape of the microneedle array 14 can be substantially any geometry. By way of non-limiting example, the microneedle array 14 can be shaped configurations including, but not limited to, irregular, square, rectangular, circular, rhomboidal, triangular, star-shaped, combinations thereof, and the like.

In various embodiments, the exterior of the microneedles 14 can have a surface coating. Suitable surface coatings include but are not limited to, antimicrobial, anticoagulant, anti-stick and the like. The coatings can range from the tip to the base 2 um to 2 cm. The thickness ranges from a few molecules to comparable to needle dimensions (1 nanometer to 10 um).

The microneedles 14 can be utilized for body fluid withdrawal and well as for injection of a fluid, which can be liquid, gas, and any flow-able medium. The depth of microneedle 14 penetration through a skin surface can vary. Preferably, the depth of penetration to provide that there is little or no pain to the patient. In this regard, it is desirable for the distal end of the microneedle 14 to breach the skin, owning for skin surface tenting effects, and travel to the capillary bed, but not extend to the distal portions of the nerve endings. Additionally, the introduction of the microneedles 14 can be controlled, via velocity control, depth of penetration, braking, and the like. As a non-limiting example, the depth of penetration, either of the microneedles 14 themselves or fluid introduction from the injector to the tissue site, can be in the range of about 100 um to 2 cm. With the present invention, the depth of penetration is selected to provide for withdrawal of one or more of, capillary blood, arterial blood, venous blood, interstitial fluid, lymphatic fluid and the like. For withdrawing capillary blood a shallower depth is used to avoid the nerve layer. At a later time, to withdraw venous blood directly from a vein, the patch 12 of the body fluid sampling/fluid delivery system 10 can be placed directly over the antecubital fossa and mid humerus. In one embodiment, the venous draw can proceed through the nerve layer with the patient experiencing some pain.

In various embodiments, the stiffness of the microneedle array 14 can vary. In one embodiment, the microneedle array 14 has sufficient rigidity to be very stiff to penetrate the skin to the selected tissue site, and sufficiently flexible to make a bend of a selected angle. In one embodiment, the bend is in the range of 0.1 to 179 degrees.

In other embodiments, the body fluid sampling/fluid delivery system 10 can include mechanisms/devices to assist in reducing the amount of pressure needed for skin penetration by the microneedle 14 or microneedle array 14. As a non-limiting example, such mechanisms/devices include but are not limited to, vibration devices such as ultrasound and mechanical vibration, electrical currents, static or dynamic penetration and the like. To help with skin penetration vibration, devices such as ultrasound and mechanical vibration, electrical currents, static or dynamic penetration can be used.

The microinjector of the present invention provides for the delivery of a fluid, such as a liquid and the like. Suitable fluids include but are not limited to, saline, an inert gas, a medicament, combinations thereof, and the like. The micro-fluidic system can be impregnated with a variety of different materials, including reagents, analyte sensors, antibodies, electrolytes, and the like.

In one embodiment, the microinjector may or may not include an outer seal to create a hermetic barrier to prevent the drop of blood from interacting with ambient air. As a non-limiting example, it is undesirable when measuring O₂ that the blood can interact with ambient air. It will be appreciated that in other tests, including but not limited to blood typing, it does not matter.

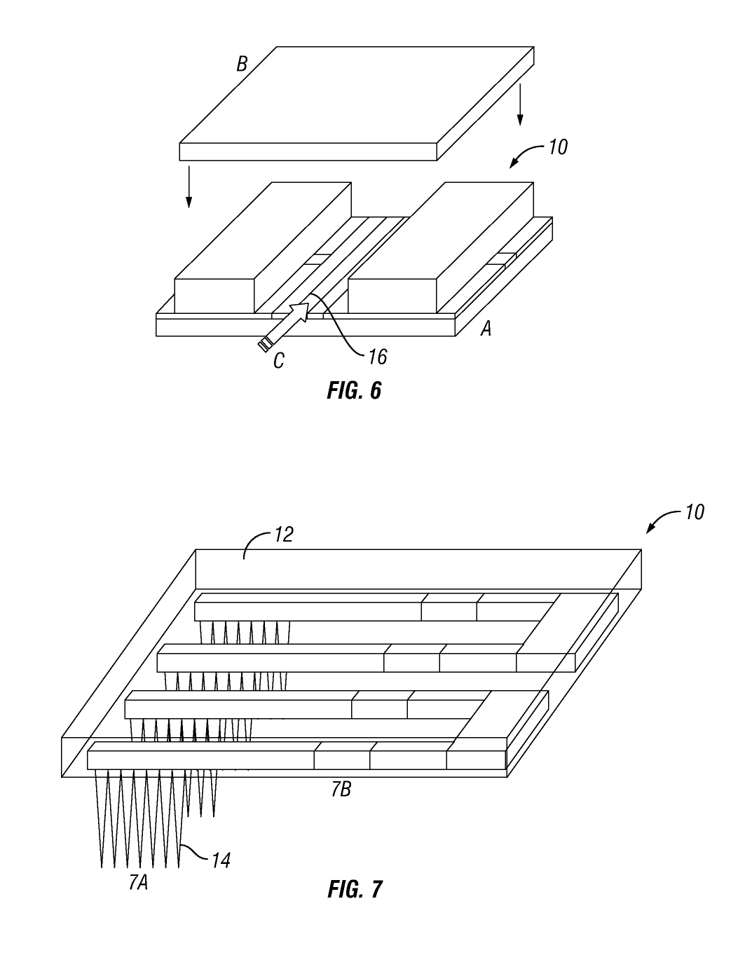

Referring now to FIG. 6, one embodiment of a microfluidic system of the present invention includes one or more microchannels 16 such as a capillary flow channel. In one embodiment, the capillary flow channel 16 is coupled to a sample chamber that houses one or more analyte sensors. Capillary forces and device backpressure result in the flow of blood through the holes of the microneedles 14 (A) into the reservoir 18 (B) the high surface to-volume ratio characteristic of this microfluidic patch 12 allows for minimal blood sampling (in the microliter range) reducing risk of iatrogenic anemia.

In one embodiment, both the capillary flow channel 16, and the sample chamber are formed as a unitary unit. The microfluidic system can be made of a variety of different materials. Additionally, the microfluidic system can be impregnated with a variety of different materials, including but not limited to reagents, analyte sensors, antibodies, electrolytes, impregnated or coated, and the like.

As a non-limiting example, a surface area and/or texture of the microchannel 16 can be optimized to propagate fluid flow in a single direction. The direction of fluid flow can be achieved by altering the texture of an interior of the microchannel 16. The microchannels 16 can be fabricated to deliver fluid in a preferred direction.

The microchannels 16 can be coated or impregnated with, or both, with a variety of different materials.

As a non-limiting example, the microneedles 14 and the microchannels 16 can be coated or impregnated with the following purified antibodies:
CD3
CD4
CD4
CD7
CD8
CD15
CD19
CD20
CD34
CD45
CD57
Cytokeratin
HLA-DR
TCR (alpha beta)
TCR (gamma delta)

### Single Color Antibodies

Bci-2
CD 16
CD1a
CD2
CD3
CD4

### ASR Reagents

Bci-2
CD 16
CD1a
CD2
CD3
CD4

### Electrolytes

In another embodiment, an electronic driver is used and coupled to the microneedle 14 or microneedle array 14, as more fully described hereafter.

FIG. 7(a) illustrates one embodiment of a microneedle 14 array. FIG. 7(b) illustrates one embodiment of a micro-machined microneedle 14 array.

In one embodiment of the present invention, polymeric materials are used for the microneedle 14 array. Polymeric microneedle 14 arrays provide a high degree of flexibility, while retaining the desirable property of stiffness, and are relatively inexpensive fabrication methods.

In one embodiment, electrodes can be embedded in the microchannel/microneedle, therefore allowing electrokinetic control and sensing of liquids and particles.

In one embodiment, the polymeric microneedle arrays 14 are made by illuminating light sensitive polymers. By way of illustration, and without limitation, ultra violet lithography, x-ray lithography and the like is used to illuminate thick layers of SU8 and PMMA to generate 3 dimensional structures. Mechanical machining, electro-discharge machining, micro-machining and micro-molding can also be used to manufacture microneedles 14. Sidewall control of the thick resist is controlled during the lithography step. Resist sidewall is sensitive to fabrication parameters such as polymer thickness, exposure dosage, clean room humidity and temperature, resist development time and the like.

In one embodiment, the polymeric microneedle arrays 14 are made by a reactive ion etch process. A reactive ion etch process involves direct targeting of a substrate by ions in an electric field. Gases such as argon can be used. As a non-limiting example, in one embodiment the microneedle 14 or microneedle array 14 are made of polymer with sharp tips coupled to microchannels 16 and the reservoirs 18. In one embodiment of the present invention, the microneedle array, microchannels 16 and reservoirs 18 are made as a monolithic multilayer structure. In another embodiment of the present invention, the microneedle array 14 is made as multiple layers that are laminated or bonded.

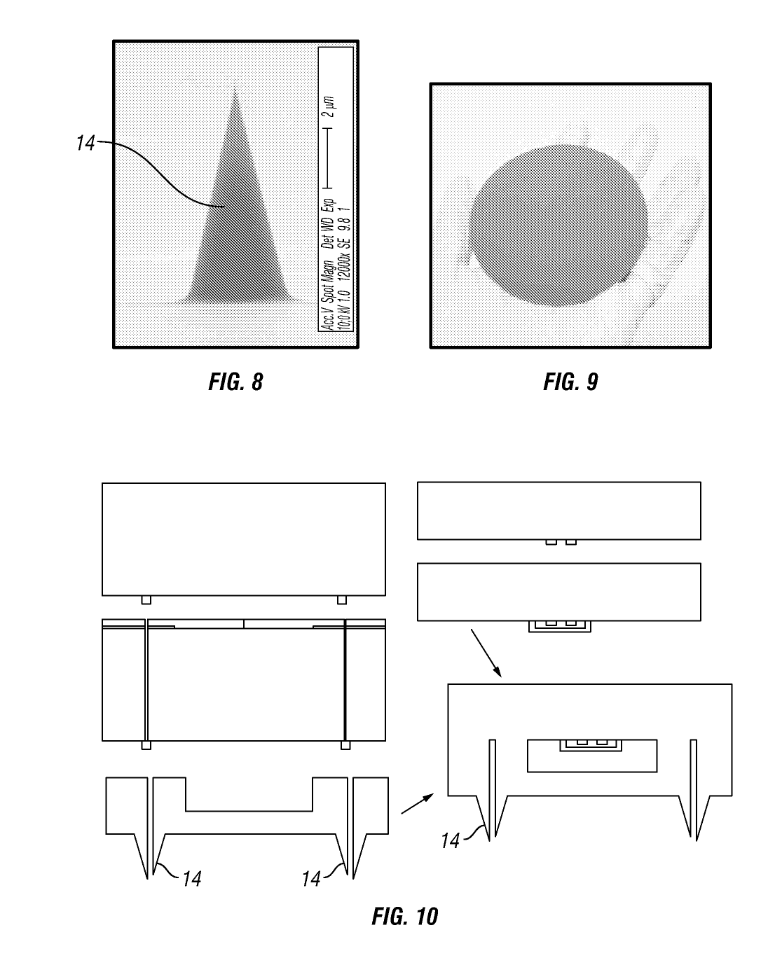

FIG. 8 illustrates one embodiment of the present invention of a silicon microneedle 14 fabricated in a top-down approach. In this embodiment, a nanometer sized photoresist pattern served as a “precursor.” The anisotropy of the structures is controlled by adjusting etch parameters. This increases the structures from nanometer size to several micrometers as the etch progressed. A highly selective, positively sloped etch is performed without undercut and the appearance of “silicon grass. The following non-limiting examples are provided without limiting the scope or nature of the present invention and are presented for illustrative purposes.

### EXAMPLE 1

In one embodiment of a mass fabrication method for microneedle array 14 formation, anisotropic reactive ion etching techniques were used with polymeric material are etched with controllable sidewall roughness and anisotropy as well as high etch mask selectivity.

The fabrication of multiple microneedles 14 was done on a wafer level, similar to the fabrication of IC chips. FIG. 9 shows a double side polished polymer wafer and etch-through holes on the wafer. A total of about 250 patches 12 on one 6″ diameter wafer were batch fabricated, providing a yield of 75%.

The fabrication of multiple microneedles 14 was done on a wafer level, similar to the fabrication of IC chips. FIG. 9 shows a double side polished polymer wafer and etch-through holes on a polymer wafer. A total of about 250 patches 12 on one 6″ diameter wafer were batch fabricated, providing a yield of 75%.

FIG. 10 shows the main batch process steps. The series of images on the left indicate the progression of the microneedle 14 layer. A virgin polyimide wafer was metal patterned on the backside using a standard lift-off lithography process. This metal layer was used as an etch mask for the microneedle 14 etch. The front of this wafer was metal patterned with two metal stacks of nm titanium and 500 nm gold. The titanium served as an etch mask for the 50 um wide vertical through holes etched. The gold was as an etch mask for the 200 um deep microchannels 16. The through holes formed the cavities in the microneedles 14 to draw the blood and the etched microchannels 16 lead the blood into the back pressure reservoir. Both etches were performed in an inductively coupled reactive ion etcher (ICP-RIE) using a gas mixture of CF4 and O2.

The series of pictures on the right of FIG. 10 show the main fabrication parts of the sensing layer and the integration of both the microneedle 14 layer and the sensing layer to form the completed patch 12. The reference electrode, green, includes an e-beam evaporated silver layer, about 1.5 μm thick, and an electrochemically fabricated silver chloride layer. The iridium oxide electrode, blue, is electrochemically plated using an IrCl4/oxalicacid/hydrogen-peroxide/potassiumcarbonate based electrolyte.

Both electrodes are placed onto a 200 μm polymer wafer (A) and then covered with the hydrogelelectrolyte, pink, which is based on poly-N-vinylpyrrolidon (PNVP). Utilization of this hydrogelelectrolyte overcomes the significant micro-fabrication challenge of storing liquid in the patch 12 by using a low melting point solid electrolyte during the fabrication of the sensor. This technique is compatible with mass manufacturing methods. The hydrogel film is conditioned with an KCl and NaOH electrolyte solution. After this treatment, the approximately 5 μm thick solid electrolyte membrane is covered with a 2 μm thick gas-permeable membrane (light blue). This membrane was formed from a silicon rubber material (SEMICOS-II). Both membranes can be deposited using the standard spin-coating method and patterned with standard photolithography.

In another embodiment, needle-free liquid jet injectors are utilized. In one embodiment, pulsed microjets are used for injection without deep penetration. As non-limiting examples, the microjets can have high velocity (v>m/s) to provide for entry of materials into the skin, small diameters as a non-limiting example 50-pm, with small volumes, which can be on the order of 2-15 nanoliters, to limit the penetration depth. The pulsed microjet injectors can be used to deliver drugs for local as well as systemic applications without using microneedles 14. The penetration depth of the microjets is controlled and limited in order to reduce tissue damage, pain and the like.

FIG. 2(b) is a schematic diagram of one embodiment of a pulsed microjet that can be used with the present invention. The pulsed microjets used with the present invention allow delivery of macromolecules, provide rapid onset, and controlled, programmable, and precise dosing, offer shallow penetration, precise injections and reduced pain and bleeding. Shallow penetration of drugs can also be advantageous for vaccination to facilitate the contact of Langerhans cells with the antigen. As a non-limiting example, the microjets can be utilized for a variety of applications including but not limited to, systemic, programmable delivery of drugs; delivery of small doses in superficial layers (for example, vaccines for immunization), and precisely local delivery into the epidermis (for example, antimicrobial agents for the treatment of acne and cold moms), and the like. The pulsed microjets use extremely small volumes and hence offer controlled delivery to superficial skin layers. In one embodiment, the microjet injector can deliver drugs at a rate of ≈1 μl/min. At a drug concentration of 20 mg/ml in the device, this flow rate translates to a delivery rate of 20 μg/min or a daily dose of ≈28 mg. This dose is sufficient for several therapeutics, including but not limited to, insulin, growth hormones, calcitonin and the like. This rate can be increased by increasing the pulsing frequency and/or using multiple nozzles. A single microjet device or an away of micronozzles can be utilized.

The microjets can be produced by displacing a desired fluid, including but not limited to a medicament, through a micronozzle by using a variety of mechanisms including but not limited to a piezoelectric transducer. Other modes of fluid displacement, include but are not limited to, piezoelectric transducer or a pressurized gas, i.e., dielectric breakdown and electromagnetic displacement, and the like.

The piezoelectric transducer, on application of a voltage pulse, expands rapidly to push a plunger that ejects the fluid from the micronozzle as a high-speed microjet. The volume of the microjet is proportional to the amplitude of the voltage pulse.

FIG. 2(b) is a schematic diagram of one embodiment of a pulsed microjet device and conventional jet injector that can be used with the present invention. The pulsed microjet injector can include a micronozzle. The micro-nozzle can be the same size as a hollow microneedle, from about 1 um to 1 mm, that can be made of a variety of materials including but not limited to an acrylic. As a non-limiting example, in one embodiment the final internal diameter can be about 50-pm into which a plunger is positioned. The plunger can be made of a variety of materials including but not limited to, stainless steel and the like. The plunger is connected to a suitable materials include but are not limited to a piezoelectric crystal and the like. The piezoelectric crystal can be activated by a pulse generator. Activation of the piezoelectric crystal pushes the plunger forward, thereby creating a microjet.

The displacement of the plunger ejects a microjet whose volume and velocity can he controlled by controlling the voltage and the rise time of the applied pulse. At the end of the stroke, the plunger is brought back to its original position. This can be achieved mechanically or with an electronic driver. In one embodiment, a compressed spring is used. As a non-limiting example, the voltage applied to the piezoelectric crystal can be varied between 0 and 140 V to generate microjets with volumes up to 15 nanoliters. The frequency of pulses cam be about 1 Hz. The fluid delivered, e.g., medicament solution, can be filled in a reservoir 18, which directly feeds the solution to the micronozzle. The reservoir 18 can be maintained at slight overpressure, a small fraction of atmospheric pressure, to avoid backflow. The solution can be degassed before loading to minimize bubble formation in some cases. As a non-limiting example, the injector can be placed against a gel or skin so that the contact was made between the two. The volume of each microjet can be measured by adding a colorimetric dye or a radiolabeled tracer, mannitol, to the solution and eject a known number of microjets. The ejected liquid can be assayed to determine the volume of each microjet.

Deactivation of the crystal moves the plunger back, and the liquid from the reservoir 18 replenishes displaced liquid. A conventional jet injector includes a nozzle into which a plunger is placed. The plunger is connected to an electro-mechanical, mechanical or compressed gas driver. By way of illustration, and without limitation, the mechanical driver can be actuated using a spring or a compressed gas chamber or electromechanical actuator.

The jet injector can be multiple or single-use devices. The disposable, single-use nozzle can be attached to a non-disposable device. As a non-limiting example, suitable operating parameters for the compressed spring and the compressed gas chamber are shown hereafter.

In another embodiment, the micronozzle is coupled to an electronic driver as described above.

Because the entire microjet ejection occurs in a fraction of a millisecond, normal bright-field microscopy by using conventional digital cameras will not capture the ejection. Frame rates of low-noise cameras under normal operation are typically no better than 50 Hz, which is very slow to be of use. To image the microjet during injection, a strobe microscopy system was used based on a fast light-emitting diode. The electronic shutter of the digital camera is turned on and a 0.31 ps flash from a light-emitting diode illuminates and freezes the jet in the image frame. A second flash delayed by a defined time using a digital delay generator (typically 5-10 μs) creates a second exposure on the same frame. From the double exposure, the average velocity between the flashes can be calculated, and a series of such images throughout the lifetime of the microjet can create a time-resolved record of the fluid ejection in air or gel.

### EXAMPLE 2

As a non-limiting example, a rise time of 10 ps lead to a mean velocity of 127 m/s for a 10-nanoliter microjet delivered from a -pm diameter micronozzle (v=Q/At, where Q is the microjet volume, A is the cross-sectional area of the micronozzle, and t is the rise time). Formation of microjets was confirmed by using high-speed photography and strobe microscopy.

By controlling the amplitude and rise time of the pulse, velocity as well as volume of the microjet was adjusted. The dispensed volume from the nozzle was replaced by liquid from a reservoir 18 that is maintained under slight positive pressure to avoid backflow.

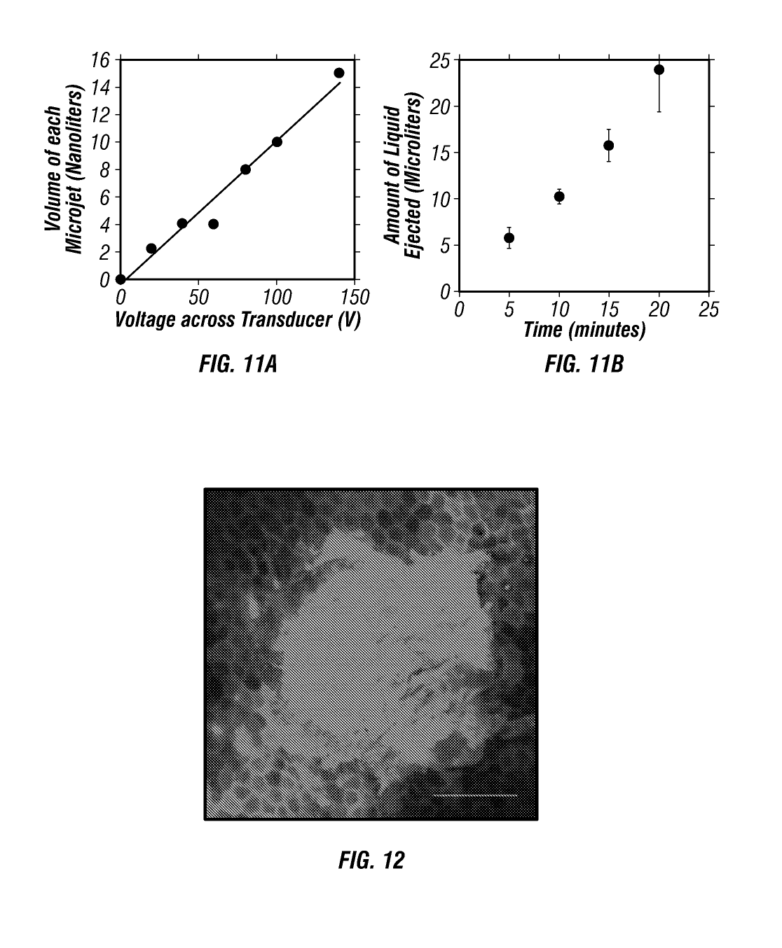

FIG. 11 illustrates one embodiment of performance characteristics of the pulsed microjet injector. As shown, there can be a dependence of microjet volume on voltage applied across the piezoelectric crystal.

### EXAMPLE 3

A microjet volume of 15 n1 was used for most experiments reported in this study. (b) Dependence of total microjet volume ejected in air as a function of time. The device was operated at a voltage of 140 V across the crystal at a frequency of I Hz, n=3; error bars correspond to SD.

Microjets were ejected from the micronozzle at exit velocities exceeding m/s and volumes of 10 to 15 nanoliters. The microjets were cylindrical in shape and each jet pulse could be clearly distinguished. To deliver volumes in excess of 10 to 15 nanoliters, the microjets were created over a prolonged period and the total amount of liquid ejected was proportional to the application time (FIG. 3b; determined with a radiolabeled tracer). For data in FIG. 3b, a pulsation frequency of 1 Hz (1 microjet per second) was used. This frequency could be increased if higher delivery rates are desired.

### EXAMPLE 4

To study the penetration of microjets into a solid substrate such as skin, a model material, agarose gel, was used. The gel offers an ideal test bed because it can be produced with controllable mechanical properties and its transparency allows direct visualization of microjet penetration. Microjets readily penetrated into agar gel, illustrated in FIG. 1(a). The penetration depth increased with increasing number of pulses. The penetration depth was established very early during the injection and stabilizes at a few millimeters after five to seven pulses. Further application of microjets did not cause substantial increase in penetration depth. Instead, the liquid delivered by microjets diffuses around the site of delivery to form a hemispherical pattern as shown in FIG. 1(bi). In the image shown in FIG. 1(bi) an estimated 35 pl of liquid was delivered into the gel by prolonged application of microjets. The diameter of the hemispherical dome in FIG. 1(bi) was about 1 cm.

FIG. 1 shows the penetration of microjets into gel and human skin in vitro with about 0.4% wt/vol agarose gel. The microjet was operated at 140 V and 1 Hz. Images represent stills from a video where, (bi) is the dispersion of dye after delivery by microjet for ≈30 min, (bii) is the penetration of a conventional jet into 0.4% wt/vol agarose gel delivered by Vitajet 3 (nozzle diameter, 177 μm; velocity >150 m/s) (injection volume of 35 μl), (c) shows the confocal microscopy pseudocolor images illustrating penetration of pulsed microjets into full-thickness human skin in vitro (1 μl/min, 1 Hz) (injection volume of 35 μl) and (d) shows optical images of penetration of conventional jet into human skin in vitro. In this example, the microjets were delivered from Vitajet 3 (nozzle diameter, 177 μm; velocity >150 m/s). (Upper) Top view. (Lower) Cross-sectional view (injection volume of 35 μl).

The difference between microjet and conventional jet injection can also be seen in human skin. Penetration depths of microjets into human skin were confirmed in vitro by using sulforhodamine B, see FIG. 1(c). Confocal microscopic analysis indicated a clear region of microjet penetration up to depths of ≈−150 μm, shown in FIG. 1(c), corresponding to a total delivery of 35 μl. Some diffused dye could be occasionally seen in the epidermis especially at long times. However, direct penetration of the microjet was not seen in deeper regions that were greater than 150 μm. Shallow penetration of microjets into skin may mitigate pain because the density of blood vessels and nerves is less in the top to 200 μm of skin.

Histologic evaluations of skin after microjet delivery showed no alterations in skin structure compared with untreated skin. However, it was difficult to reach a conclusion based on these data because it was not clear whether the actual injection site was captured in the histology section. The microjet itself is ≈pm in diameter and penetrates ≈−150 μm into skin. Experiments with confocal microscopy provided information about the tissue structure adjacent to the microinjection site as illustrated in FIG. 12. This image, taken ≈15-30 min postinjection, shows the injection spot, the bright circular region, and the hexagonal architecture of corneocytes around the injection spot stained by the dye, which diffused from the injection site. The architecture of corneocytes appears intact and suggests that microjet penetration has no adverse effect on tissue morphology adjacent to the injection site. The tissue structure within the actual site of microjet penetration is likely to be altered as a result of compression and shear-induced damage after microjet impact and entry. However, these alterations are local and superficial within the penetration region of a few hundred microns. These structural may be reversible as a result of a combined effect of skin's elasticity, barrier recovery processes, and ultimately, epidermal turnover.

FIG. 12 is an image that shows the penetration of microjets into human skin in vitro, and more particularly, the intact structure of corneocytes around an injection site which is the bright spot at the center. The image was taken 15-30 min postinjection. (Scale bar, 200 μm.)

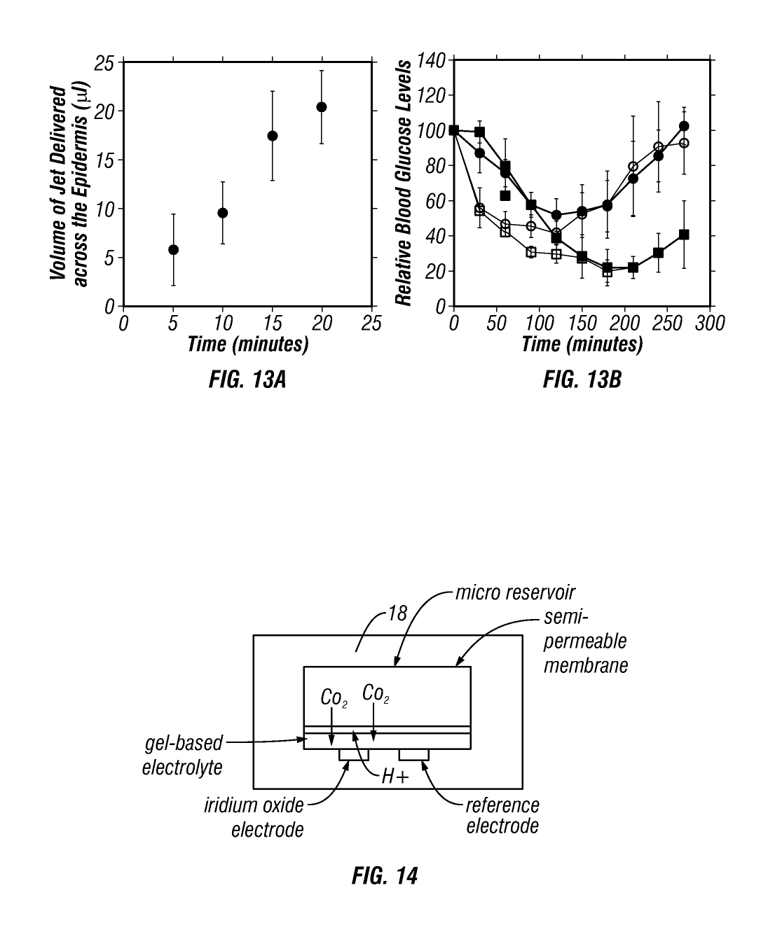

Quantitative estimates of microjet penetration into human skin were obtained by using radiolabeled mannitol as a tracer. For this purpose, a separate model system was designed in which isolated human epidermis was placed on the agarose gel and microjets containing a colorimetric dye and radiolabeled mannitol were delivered. Visual appearance of the dye in the gel was used to determine the number of pulses necessary to penetrate the epidermis, whereas quantitative determination of the amount of liquid delivered across the epidermis was obtained by using mannitol. A single pulse was not sufficient to penetrate the epidermis. The median number of pulses required for visible appearance of the dye across the epidermis was 48. This corresponds to a median penetration time of 48 seconds when microjets were delivered at a rate of 1 Hz. This can be reduced by up to 10-fold by increasing the microjet delivery rate to 10 Hz. During this short lag time, a negligible amount of mannitol was detected in the supporting gel. Beyond this period, the amount of mannitol delivered increased linearly with time, as shown in FIG. 13(a). The rate of transdermal mannitol delivery under the conditions shown in FIG. 13(a) is =1 μl/min.

FIG. 13 illustrates the transdermal delivery of mannitol in human skin in vitro and insulin in rat in vivo. (a) Penetration of microjets across human epidermis in vitro (1 μl/min; 1 Hz). Penetration increases linearly with time (n=3; error bars show SD). (b) Delivery of insulin in Sprague-Dawley rats in vivo (1 μl/min, 1 Hz). Filled squares, microjets delivered for 20 min; filled circles, microjets delivered for 10 min; open circles, s.c. injection of 1.5 units; open squares, conventional jet injection (Vitajet 3, 2 units) (n=3-5; error bars correspond to SD).

### EXAMPLE 5

As shown in Sprague-Dawley rats using insulin as the model drug. The animals were put under anesthesia (1-4% isoflurane) and rested on their back during the procedure. The hair on the abdomen were lightly shaved for placement of the injector orifice close to the skin while avoiding any damage to skin. The orifice of the microjet was placed against the skin, thus ensuring minimal standoff distance and mimicking use of traditional jet injectors in humans. Insulin solution (Sigma-Aldrich) with activity of units/ml was delivered for 10 or 21) min and blood samples collected from the tail vein before the start of injection and every 30 min thereafter. Sample collection was continued for 2 min after initiation of insulin delivery and all samples were immediately assayed for glucose level by One Touch glucose meter (LifeScan, Inc., Milpitas, Calif.). s.c. injection of 1.5 units served as a positive control. As an additional control, 2 units insulin was delivered using a commercial jet injector (Vitajet 3; Bioject, Inc.). All experiments were performed under protocols approved by the Institutional Animal Care and Use Committee.

Microjet-delivered insulin was rapidly absorbed into systemic circulation as evidenced by a rapid decrease in blood glucose levels in a dose-dependent manner (FIG. 13, closed squares, 20-min delivery; and closed circles, 10-min delivery). As a positive control, 1.5 units insulin was injected s.c. (FIG. 13, open circles). Under the microjet parameters used in these experiments, it is anticipated that 2 units of insulin was delivered over 20 min, and 1 unit was delivered in 10 min (delivery of units/ml insulin at ≈1 μl/min). A proportional reduction in glucose levels was observed when microjets were delivered for 10 and 20 min (the area above the 10-min curve in FIG. 13 b is 56% of that above the 20-min curve). The drop in glucose levels was faster with s.c. injection. However, the area above the s.c. injection curve was comparable to the average numbers for microjet injections of 1 and 2 units, indicating the bioequivalence of the two methods. As another positive control, 2 units insulin were delivered with a conventional jet injector (Vitajet 3, open squares). The conventional injector induced significantly rapid hypoglycemia compared with microjets, possibly as a result of deeper and wider penetration. However, jet injections were associated with significant adverse effects. Significant bleeding was observed in one animal and severe erythema was observed in another animal. No adverse effects, bleeding or erythema, were observed at the site of microjet injection. The site of injection itself did not have any visible mark after delivery. This is attributed to superficial penetration of microjets into skin.

### EXAMPLE 6

A blood gas, including but not limited to carbon dioxide concentration, was measured in a reservoir 18 and is based on the Severinghaus principle. Its original structures consist of a reference electrode, a pH glass electrode filled with liquid, an electrolyte solution and a hydrophobic gas permeable membrane. Numerous miniaturized versions of the electrodes have been proposed utilizing the basic operation of the Severinghaus electrode. These include the optode, ISFET, and the application of the liquid-membrane electrode.

Electrochemically grown iridium oxide films (EIROF) were used as the pH sensing element. EIROF is highly sensitive to pH, has a fast response time, exhibits little drift and has a long lifetime.

The operation principle is indicated in FIG. 14. As the blood sample traveled through the microchannel 16 into the sensor part, the CO2 diffused through a gas-permeable membrane into the electrolyte. It under went hydration and formed carbonic acid and bicarbonate, that subsequently formed free hydrogen. The electrolyte was prepared such that the change of pH inside the electrolyte was proportional to the CO2 concentration in the blood. This change generated a characteristic potential between the iridium oxide electrode and the reference electrode, indicating the CO2 concentration.

The sensing mechanisms for the different blood gas parameters (O2, CO2 and pH) are very similar in their fabrication methodology and their functionality.

For the preceding examples, the gel was prepared on the day of use by dissolving agarose (Sigma Aldrich Corp, St. Louis, Mo.) in deionized water. The microjet system was loaded with degassed saline mixed with blue dye. Microjet injections were carried out at constant frequency of 1 Hz in 0.4% agarose gel for up to min. Images of microjets penetrating into gels were obtained by using a digital camera (Optronics, Goleta, Calif.).

Human skin was obtained from the National Disease Research Interchange (NDRI, Philadelphia, Pa.). Epidermis was separated from full-thickness skin by using standard procedures and was placed on 0.4% agarose gel. The microjet injector was loaded with degassed saline mixed with 50 iCi/m1 314-labeled mannitol (American Radiolabeled Chemicals, Inc., St. Louis, Mo.) and 10 mM sulforhodamine B (Molecular Probes, Eugene, Oreg.). Delivery across epidermis was quantified by visually confirming appearance of the dye in the gel and by measuring the amount of radioactivity in gel. For this purpose, the gel was collected at various time points in separate experiments and dissolved in Solvable tissue solubilizer (Perkin-Elmer Life and Analytical Sciences, Inc., Boston, Mass.). Radioactivity was counted by using Packard Tri-Garb 2TR Scintillation Counter (Packard, Meridien, Conn.).

Penetration of microjets into human skin was assessed by using confocal microscopy. Full-thickness human skin was used for this purpose. Microjet injector was loaded with 10 lnM sulforhodamine B (Molecular Probes, Eugene, Oreg.) in degassed saline. The injector was placed on the skin and activated for 5-35 min at a frequency of 1 Hz. The skin sample was mounted on glass slide and immediately frozen until analysis to prevent diffusion of the dye. Depth and dispersion pattern of injections were visualized by using confocal microscope (Leica Microsystems, Bannockburn, Ill.). The samples were excited at 5 nm and emission spectra captured between 5 and 0 nat. Images were obtained in Ay: scanning mode and captured every 2 min from the skin surface until no appreciable fluorescence could be detected. Each image represents an average of two scans.

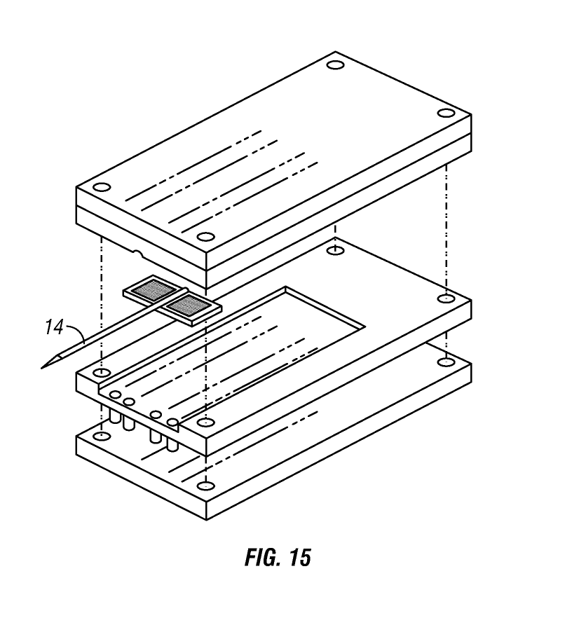

Referring to FIG. 15 a controllable electronic driver, which can be an electromagnetic driver, can be used to drive the microneedle 14 or microneedle array 14. The term electromagnetic driver, as used herein, generally includes any device that moves or drives the microneedle 14 or microneedle array 14 under an electrically or magnetically induced force. FIG. 13 is a partially exploded view of an embodiment of an electromagnetic driver. The top half of the driver is shown assembled. The bottom half of the driver is shown exploded for illustrative purposes.

FIG. 15 shows an inner insulating housing separated from a stationary housing or PC board, and the microneedle 14 or microneedle array 14 and flag assembly separated from the inner insulating housing for illustrative purposes. In an embodiment, each coil drive field core in the PC board located in the PC Board and 30 is connected to the inner insulating housing with rivets.

In one embodiment, the electromagnetic driver has a magnetically permeable flag attached at the proximal or drive end and a stationary part comprising a stationary housing assembly with electric field coils arranged so that they produce a balanced field at the flag to reduce or eliminate any net lateral force on the flag. The electric field coils are generally one or more metal coils, which generate a magnetic field when electric current passes through the coil. The iron flag is a flat or enlarged piece of magnetic material to enhance the magnetic forces generated between a microneedle 14 or microneedle array 14 and a magnetic field produced by the field coils. The combined mass of the microneedle 14 or microneedle array 14 and the iron flag can be minimized to facilitate rapid acceleration for introduction into the skin of a patient, to reduce the impact when the microneedle 14 or microneedle array 14 stops in the skin, and to facilitate prompt velocity profile changes throughout the sampling cycle.

The stationary housing assembly can include a PC board, a lower inner insulating housing, an upper inner insulating housing, an upper PC board, and rivets assembled into a single unit.

The electric field coils in the upper and lower stationary housing and 30 are fabricated in a multi-layer printed circuit (PC) board. They may also be conventionally wound wire coils. A Teflon® material, or other low friction insulating material is used to construct the lower and upper inner insulating housing. Each insulating housing is mounted on the PC board to provide electrical insulation and physical protection, as well as to provide a low-friction guide for the microneedle 14 or microneedle array 14. The lower and upper inner insulating housing provide a reference surface with a small gap so that the microneedle 14 or microneedle array 14 can align with the drive field coils in the PC board for good magnetic coupling.

Rivets connect the lower inner insulating housing to the lower stationary housing and are made of magnetically permeable material such as ferrite or steel, which serves to concentrate the magnetic field. This mirrors the construction of the upper inner insulating housing and upper stationary housing 30. These rivets form the poles of the electric field coils. The PC board is fabricated with multiple layers of coils or with multiple boards. Each layer supports spiral traces around a central hole. Alternate layers spiral from the center outwards or from the edges inward. In this way each layer connects via simple feed-through holes, and the current always travels in the same direction, summing the ampere-turns.

The PC boards within the lower and upper stationary housings and are connected to the lower and upper inner insulating housings and with the rivets . The lower and upper inner insulating housings and expose the rivet heads on opposite ends of the slot where the microneedle 14 or microneedle array 14 travels. The magnetic field lines from each rivet create magnetic poles at the rivet heads. An iron bar on the opposite side of the PC board within each of the lower and upper stationary housing and completes the magnetic circuit by connecting the rivets. Any fastener made of magnetically permeable material such as iron or steel can be used In place of the rivets. A single component made of magnetically permeable material and formed in a horseshoe shape can be used in place of the rivet/screw and iron bar assembly. In operation, the magnetically permeable flag attached to the microneedle 14 or microneedle array 14 is divided into slits and bars. The slit patterns are staggered so that coils can drive the flag in two, three or more phases.

Both lower and upper PC boards and contain drive coils so that there is a symmetrical magnetic field above and below the flag. When the pair of PC boards is turned on, a magnetic field is established around the bars between the slits of the magnetically permeable iron on the flag. The bars of the flag experience a force that tends to move the magnetically permeable material to a position minimizing the number and length of magnetic field lines and conducting the magnetic field lines between the magnetic poles.

When a bar of the flag is centered between the rivets of a magnetic pole, there is no net force on the flag, and any disturbing force is resisted by imbalance in the field. This embodiment of the device operates on a principle similar to that of a solenoid. Solenoids cannot push by repelling iron; they can only pull by attracting the iron into a minimum energy position. The slits on one side of the flag are offset with respect to the other side by approximately one half of the pitch of the poles. By alternately activating the coils on each side of the PC board, the microneedle 14 or microneedle array 14 can be moved with respect to the stationary housing assembly. The direction of travel is established by selectively energizing the coils adjacent the metal flag on the microneedle 14 or microneedle array 14. Alternatively, a three phase, three-pole design or a shading coil that is offset by one-quarter pitch establishes the direction of travel. The lower and upper PC boards and shown in FIG. 13 contain electric field coils, which drive the microneedle 14 or microneedle array 14 and the circuitry for controlling the entire electromagnetic driver.

The embodiment described above generally uses the principles of a magnetic attraction drive, similar to commonly available circular stepper motors (Hurst Manufacturing BA Series motor, or “Electrical Engineering Handbook” Second edition p 1472-1474, 1997). These references are hereby incorporated by reference. Other embodiments can include a linear induction drive that uses a changing magnetic field to induce electric currents in the microneedle 14 or microneedle array 14. These induced currents produce a secondary magnetic field that repels the primary field and applies a net force on the microneedle 14 or microneedle array 14. The linear induction drive uses an electrical drive control that sweeps a magnetic field from pole to pole, propelling the microneedle 14 or microneedle array 14 before it. Varying the rate of the sweep and the magnitude of the field by altering the driving voltage and frequency controls the force applied to the microneedle 14 or microneedle array 14 and its velocity.

The arrangement of the coils and rivets to concentrate the magnetic flux also applies to the induction design creating a growing magnetic field as the electric current in the field switches on. This growing magnetic field creates an opposing electric current in the conductive flag. In a linear induction motor the flag is electrically conductive, and its magnetic properties are unimportant. Copper or aluminum are materials that can be used for the conductive flags. Copper is generally used because of its good electrical conductivity. The opposing electrical field produces an opposing magnetic field that repels the field of the coils. By phasing the power of the coils, a moving field can be generated which pushes the flag along just below the synchronous speed of the coils. By controlling the rate of sweep, and by generating multiple sweeps, the flag can be moved at a desired speed.

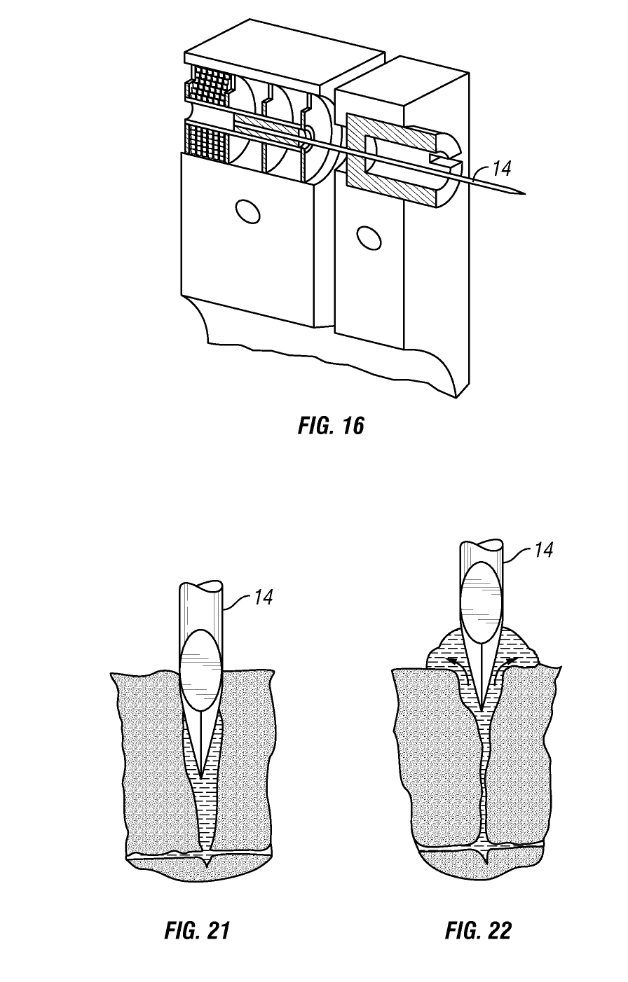

FIG. 16 shows another embodiment of a solenoid type electromagnetic driver that is capable of driving an iron core or slug mounted to the microneedle 14 or microneedle array 14 using a direct current (DC) power supply. The electromagnetic driver includes a driver coil pack that is divided into three separate coils along the path of the microneedle 14 or microneedle array 14, two end coils and a middle coil. Direct current is alternated to the coils to advance and retract the microneedle array 14 or microneedle array 14. Although the driver coil pack is shown with three coils, any suitable number of coils may be used, for example, 4, 5, 6, 7 or more coils may be used.

The stationary iron housing contains the driver coil pack with a first coil is flanked by iron spacers which concentrate the magnetic flux at the inner diameter creating magnetic poles. The inner insulating housing 48 isolates the microneedle 14 or microneedle array 14 and iron core from the coils and provides a smooth, low friction guide surface. The microneedle 14 or microneedle array guide further centers the microneedle 14 or microneedle array 14 and iron core. The microneedle 14 or microneedle array 14 is protracted and retracted by alternating the current between the first coil 52, the middle coil, and the third coil to attract the iron core. Reversing the coil sequence and attracting the core and microneedle 14 or microneedle 14 array back into the housing retracts the microneedle 14 or microneedle array 14. The microneedle 14 or microneedle array guide also serves as a stop for the iron core mounted to the microneedle 14 or microneedle array 14.

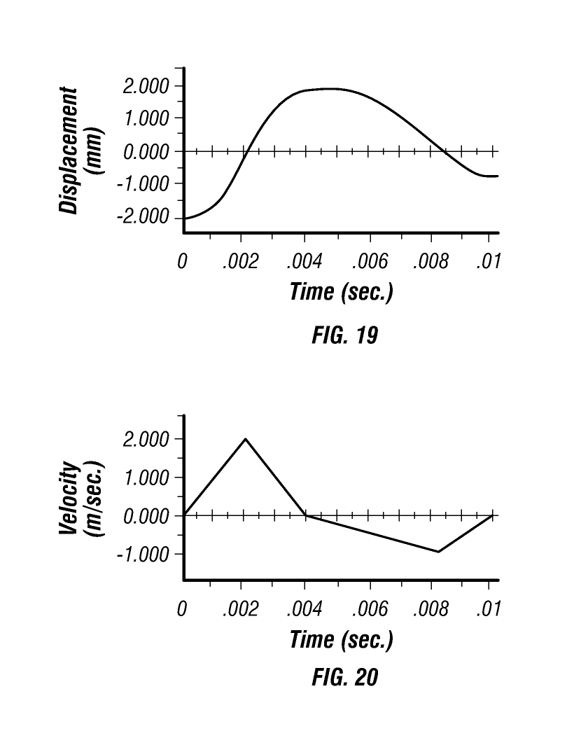

Penetration devices which employ spring or cam driving methods have a symmetrical or nearly symmetrical actuation displacement and velocity profiles on the advancement and retraction of the microneedle 14 or microneedle array 14 as shown in FIGS. 19 and 20. In most of once the launch is initiated, the stored energy determines the velocity profile until the energy is dissipated. Controlling impact, retraction velocity, and dwell time of the microneedle 14 or microneedle array 14 within the tissue can be useful in order to achieve a high success rate, while accommodating variations in skin properties and minimize pain. Advantages can be achieved by taking into account that tissue dwell time is related to the amount of skin deformation as the microneedle 14 or microneedle array 14 tries to puncture the surface of the skin and variance in skin deformation from patient to patient based on skin hydration.

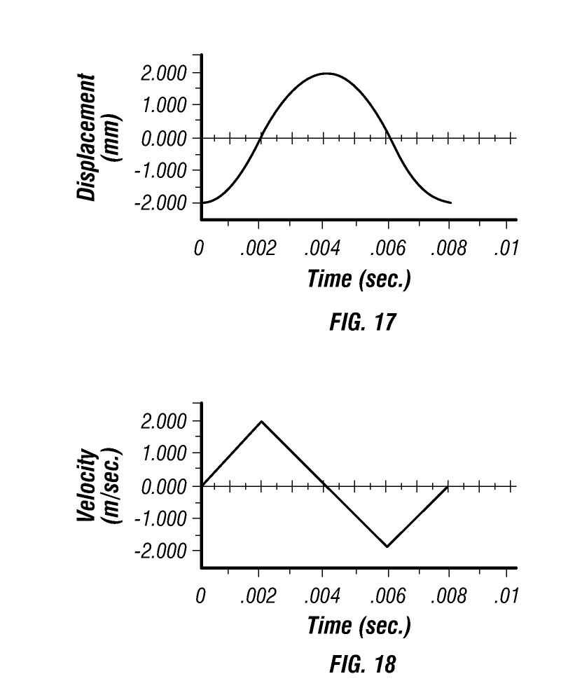

The ability to control velocity and depth of penetration can be achieved by use of a controllable force driver where feedback is an integral part of driver control. The dynamic control of such a driver is illustrated in FIG. 19 which illustrates an embodiment of a controlled displacement profile and FIG. 20 which illustrates an embodiment of a the controlled velocity profile. These are compared to FIGS. 17 and 18, which illustrate embodiments of displacement and velocity profiles, respectively, of a harmonic spring/mass powered driver.

Reduced pain can be achieved by using impact velocities of greater than 2 m/s entry of the microneedle 14 or microneedle array 14.

Retraction of the microneedle 14 or microneedle array 14 at a low velocity following the sectioning of the venuole/capillary mesh allows the blood to flood the wound tract and flow freely to the surface, thus using the microneedle 14 or microneedle array 14 to keep the microchannel 16 open during retraction as shown in FIGS. 17 and 22. Low-velocity retraction of the microneedle 14 or microneedle array 14 near the wound flap prevents the wound flap from sealing off the microchannel 16. Thus, the ability to slow the microneedle 14 or microneedle array 14 retraction directly contributes to increasing the success rate of obtaining blood. Increasing the sampling success rate to near 100% can be important to the combination of sampling and acquisition into an integrated sampling module such as an integrated glucose-sampling module, which incorporates a glucose test strip.

Referring again to FIG. 17, the microneedle 14 or microneedle array 14 and microneedle 14 or microneedle array 14 driver are configured so that feedback control is based on microneedle 14 or microneedle array 14 displacement, velocity, or acceleration. The feedback control information relating to the actual microneedle 14 or microneedle array 14 path is returned to a processor such as that illustrated in FIG. 22 that regulates the energy to the driver, thereby precisely controlling the microneedle 14 or microneedle array 14 throughout its advancement and retraction. The driver may be driven by electric current, which includes direct current and alternating current.

In FIG. 17, the electromagnetic driver shown is capable of driving an iron core or slug mounted to the microneedle 14 or microneedle array 14 using a direct current (DC) power supply and is also capable of determining the position of the iron core by measuring magnetic coupling between the core and the coils. The coils can be used in pairs to draw the iron core into the driver coil pack. As one of the coils is switched on, the corresponding induced current in the adjacent coil can be monitored. The strength of this induced current is related to the degree of magnetic coupling provided by the iron core, and can be used to infer the position of the core and hence, the relative position of the microneedle 14 or microneedle array 14.

After a period of time, the drive voltage can be turned off, allowing the coils to relax, and then the cycle is repeated. The degree of magnetic coupling between the coils is converted electronically to a proportional DC voltage that is supplied to an analog-to-digital converter. The digitized position signal is then processed and compared to a desired “nominal” position by a central processing unit (CPU). The CPU to set the level and/or length of the next power pulse to the solenoid coils uses error between the actual and nominal positions.

In another embodiment, the driver coil pack has three coils consisting of a central driving coil flanked by balanced detection coils built into the driver assembly so that they surround an actuation or magnetically active region with the region centered on the middle coil at mid-stroke. When a current pulse is applied to the central coil, voltages are induced in the adjacent sense coils. If the sense coils are connected together so that their induced voltages oppose each other, the resulting signal will be positive for deflection from mid-stroke in one direction, negative in the other direction, and zero at mid-stroke. This measuring technique is commonly used in Linear Variable Differential Transformers (LVDT). Microneedle 14 or microneedle array 14 position is determined by measuring the electrical balance between the two sensing coils.

In another embodiment, a feedback loop can use a commercially available LED/photo transducer module such as the OPB703 manufactured by Optek Technology, Inc., 1215 W. Crosby Road, Carrollton, Tex., 75006 to determine the distance from the fixed module on the stationary housing to a reflective surface or target mounted on the microneedle 14 or microneedle array 14. The LED acts as a light emitter to send light beams to the reflective surface, which in turn reflects the light back to the photo transducer, which acts as a light sensor. Distances over the range of 4 mm or so are determined by measuring the intensity of the reflected light by the photo transducer. In another embodiment, a feedback loop can use a magnetically permeable region on the microneedle 14 or microneedle array 14 itself as the core of a Linear Variable Differential Transformer (LVDT).

A permeable region created by selectively annealing a portion of the microneedle 14 or microneedle array 14, or by including a component in the microneedle 14 or microneedle array 14, such as ferrite, with sufficient magnetic permeability to allow coupling between adjacent sensing coils. Coil size, number of windings, drive current, signal amplification, and air gap to the permeable region are specified in the design process. In another embodiment, the feedback control supplies a piezoelectric driver, superimposing a high frequency oscillation on the basic displacement profile. The piezoelectric driver provides improved cutting efficiency and reduces pain by allowing the microneedle 14 or microneedle array 14 to “saw” its way into the tissue or to destroy cells with cavitation energy generated by the high frequency of vibration of the advancing edge of the microneedle 14 or microneedle array 14. The drive power to the piezoelectric driver is monitored for an impedance shift as the device interacts with the target tissue. The resulting force measurement, coupled with the known mass of the microneedle 14 or microneedle array 14 is used to determine microneedle 14 or microneedle array 14 acceleration, velocity, and position.

The body fluid sampling/fluid delivery system 10 can include a user interface or a display configured to relay different information, including but not limited to, skin penetrating performance, a skin penetrating setting, and the like. Display can provide a user with at a variety of different outputs, including but not limited to, penetration depth of a microneedle 14 or microneedle array 14, velocity of a microneedle 14 or microneedle array 14, a desired velocity profile, a velocity of microneedle 14 or microneedle array 14 into target tissue, velocity of the microneedle 14 or microneedle array 14 out of target tissue, dwell time of microneedle 14 or microneedle array 14 in target tissue, a target tissue relaxation parameter, and the like. Display can include a variety of components including but not limited to, a real time clock, one or more alarms to provide a user with a reminder of a next target penetrating event is needed, a user interface the processor, and the like.

The display can play a passive role and merely display results, or be more active. Display can provide a variety of different outputs to a user including but not limited to, actual depth of microneedle 14 or microneedle array 14 penetration on target tissue, stratum corneum thickness in the case where the target tissue is the skin and an area below the skin, force delivered on target tissue, energy used by a microneedle 14 or microneedle array 14 driver to drive a microneedle 14 or microneedle array 14 into target tissue, dwell time of microneedle 14 or microneedle array 14, battery status of the body fluid sampling/fluid delivery system 10, status of the body fluid sampling/fluid delivery system 10, the amount of energy consumed by the body fluid sampling/fluid delivery system 10 or any component of the body fluid sampling/fluid delivery system 10, speed profile of microneedle 14 or microneedle array 14, information relative to contact of microneedle 14 or microneedle array 14 with target tissue before penetration by microneedle 14 or microneedle array 14, information relative to a change of speed of microneedle 14 or microneedle array 14 as it advances in target tissue, and the like.

Display can include a data interface that couples body fluid sampling/fluid delivery system 10 to support equipment with an interface, the internet, and the like. The data interface may also be coupled to the processor 93. Suitable support equipment includes but is not limited to, a base station, home computer, central server, main processing equipment for storing analyte, such as glucose, level information, and the like.

Data interface can be a variety of interfaces including but not limited to, Serial RS-232, modem interface, USB, HPNA, Ethernet, optical interface, IRDA, RF interface, BLUETOOTH interface, cellular telephone interface, two-way pager interface, parallel port interface standard, near field magnetic coupling, RF transceiver, telephone system, and the like.

Display be coupled to a the memory that stores, a target tissue parameter, target tissue penetrating performance, and the like. The memory may also be connected to a processor and store data from the user interface.

In one embodiment, the memory can store, the number of target tissue penetrating events, time and date of the last selected number of target tissue penetrating events, time interval between alarm and target tissue penetrating event, stratum corneum thickness, time of day, depth of microneedle 14 or microneedle array 14 penetration, velocity of microneedle 14 or microneedle array 14, a desired velocity profile, velocity of microneedle 14 or microneedle array 14 into target tissue, velocity of microneedle 14 or microneedle array 14 out of target tissue, dwell time of microneedle 14 or microneedle array 1.4 in target tissue, a target tissue relaxation parameter, force delivered on target tissue by any component of the body fluid sampling/fluid delivery system 10, dwell time of microneedle 14 or microneedle array 14, battery status of body fluid sampling/fluid delivery system 10, body fluid sampling/fluid delivery system 10 status, consumed energy by body fluid sampling/fluid delivery system 10 or any of its components, speed profile of microneedle 14 or microneedle array 14 as it penetrates and advances through target tissue, a tissue target tissue relaxation parameter, information relative to contact of microneedle 14 or microneedle array 14 with target tissue before penetration by microneedle 14 or microneedle array 14, information relative to a change of speed of microneedle 14 or microneedle array 14 as in travels in and through target tissue. In one embodiment, the processor is coupled to and receives any of a different type of signals from user interface. Display can respond to a variety of different commands, including but not limited to audio commands, and the like. Display can include a sensor for detecting audio commands. Information can be relayed to a user of body fluid sampling/fluid delivery system 10 by way of an audio device, wireless device, and the like.

In another embodiment, the body fluid sampling/fluid delivery system 10 includes a human interface with at least one output. The human interface is specific for use by humans while a display may be for any type of user, with user defined generically. Human interface can be coupled to the processor and a body fluid sampling/fluid delivery system 10 sensor. Human interface can be a variety of different varieties including but not limited to, LED, LED digital display, LCD display, sound generator, buzzer, vibrating device, and the like.

The output of human interface can be a variety of outputs including but not limited to, a penetration event by microneedle 14, time of day, alarm, microneedle 14 or microneedle array 14 trajectory waveform profile information, force of last penetration event, last penetration event, battery status of the body fluid sampling/fluid delivery system 10, analyte or injected fluid status, time to change cassette status, jamming malfunction, body fluid sampling/fluid delivery system 10 status, and the like.

Human interface is coupled to a housing. Suitable housings include but are not limited to a, telephone, watch, PDA, electronic device, medical device, point of care device, decentralized diagnostic device and the like. An input device is coupled to housing. Suitable input devices include but are not limited to, one or more pushbuttons, a touch pad independent of the display device, a touch sensitive screen on a visual display, and the like.

A data exchange device can be utilized for coupling body fluid sampling/fluid delivery system 10 to support equipment including but not limited to, personal computer, modem, PDA, computer network, and the like. Human interface can include a real time clock, and one or more alarms that enable a user to, set and use for reminders for the next target tissue penetration event. Human interface can be coupled to a human interface the processor which is distinct from the processor. Human interface the processor can include a sleep mode and can run intermittently to conserve power. Human interface the processor includes logic that can provide an alarm time set for a first subset of days, and a second alarm time set for a second subset of days. By way of example, and without limitation, the first subset of days can be Monday through Friday, and the second subset of days can be Saturday and Sunday.

Human interface can be coupled to a the memory for storing a variety of information, including but not limited to, the number of target tissue penetrating events, time and date of the last selected number of target tissue penetrating events, time interval between alarm and target tissue penetrating event, stratum corneum thickness when target tissue is below the skin surface and underlying tissue, time of day, depth of microneedle 14 or microneedle array 14 penetration, velocity of microneedle 14 or microneedle array 14, a desired velocity profile, velocity of microneedle 14 or microneedle array 14 into target tissue, velocity of microneedle 14 or microneedle array 14 out of target tissue, dwell time of microneedle 14 or microneedle array 14 in target tissue, a target tissue relaxation parameter, force delivered on target tissue, dwell time of microneedle 14 or microneedle array 14, battery status of body fluid sampling/fluid delivery system 10 and its components, body fluid sampling/fluid delivery system 10 status, consumed energy, speed profile of microneedle 14 or microneedle array 14 as it advances through target tissue, a target tissue relaxation parameter, information relative to contact of a microneedle 14 or microneedle array 14 with target tissue before penetration by microneedle 14 or microneedle array 14, information relative to a change of speed of microneedle 14 or microneedle array 14 as in travels in target tissue, information relative to consumed sensors.

The operation of a feedback loop that can be used with the body fluid sampling/fluid delivery system 10 of the present invention, as well as a processor. The processor can store tissue penetration information, patient information, information regarding microneedle 14 velocity, and the like, in a non-volatile memory. In one embodiment, inputs are provided about the desired circumstances or parameters for a tissue penetration. The processor selects a profile from a set of alternative profiles are preprogrammed in the processor based on typical or desired body fluid sampling/fluid delivery system 10 performance determined through testing at the factory, as programmed in by the operator and the like. The processor may customize by either scaling or modifying the profile based on additional user input information. Once the processor has chosen and customized the profile, the processor is ready to modulate the power from a power supply to the microneedle 14 driver through an amplifier. The processor may measure the location of the microneedle 14 or microneedle array 14 using a position sensing mechanism through an analog to digital converter linear encoder or other such transducer. A microneedle 14 position sensor can be provided.

The processor calculates the movement of the microneedle 14 or microneedle array 14 by comparing the actual profile of the microneedle 14 or microneedle array 14 to the predetermined profile. The processor modulates the power to the microneedle/microneedle array 14 driver through a signal generator, which may control the amplifier so that the actual velocity profile of the microneedle 14 or microneedle array 14 does not exceed the predetermined profile by more than a preset error limit. The error limit is the accuracy in the control of the microneedle 14 or microneedle array 14.

After the microneedle 14 penetration or fluid delivery event, the processor can allow the user to rank the results of the microneedle 14 penetration or fluid delivery event. The processor stores these results and constructs a database for the individual user. Using the database, the processor calculates the profile traits such as degree of painlessness, success rate, and blood volume for various profiles depending on user input information to optimize the profile to the individual user for subsequent microneedle 14 penetration or fluid delivery cycles. These profile traits depend on the characteristic phases of microneedle 14 or microneedle array 14 advancement and retraction.

The processor uses these calculations to optimize profiles for each user. In addition to user input information, an internal clock allows storage in the database of information such as the time of day to generate a time stamp for the microneedle 14 penetration or fluid delivery event and the time between microneedle 14 penetration or fluid delivery events to anticipate the user's diurnal needs. The database stores information and statistics for each user and each profile that particular user uses.

In addition to varying the profiles, the processor can be used to calculate the appropriate microneedle 14 or microneedle array 14 diameter and geometry suitable to realize the blood volume required by the user. For example, if the user requires about 1-5 microliter volume of blood, the processor may select a 200 um diameter microneedle 14 or microneedle array 14 to achieve these results. For each class of microneedle 14 or microneedle array 14, both diameter and microneedle 14 or microneedle array 14 tip geometry, is stored in the processor to correspond with upper and lower limits of attainable blood volume based on the predetermined displacement and velocity profiles.

The body fluid sampling/fluid delivery system 10 is capable of prompting the user for information at the beginning and the end of the microneedle 14 penetration or fluid delivery event to more adequately suit the user. The goal is to either change to a different profile or modify an existing profile. Once the profile is set, the force driving the microneedle 14 or microneedle array 14 is varied during advancement and retraction to follow the profile. The method of microneedle 14 penetration or fluid delivery using the body fluid sampling/fluid delivery system 10 comprises selecting a profile, microneedle 14 penetration or fluid delivery according to the selected profile, determining microneedle 14 penetration or fluid delivery profile traits for each characteristic phase of the microneedle 14 penetration or fluid delivery cycle, and optimizing profile traits for subsequent microneedle 14 penetration or fluid delivery events.

In another embodiment, the microneedle 14 penetration or fluid delivery system 10 includes a controllable driver coupled to a microneedle 14 or microneedle array 14. The body fluid sampling/fluid delivery system 10 has a proximal end and a distal end. At the distal end is the tissue penetration element in the form of the microneedle 14 or microneedle array 14, which is coupled to an elongate coupler shaft by a drive coupler. The elongate coupler shaft has a proximal end and a distal end. A driver coil pack is disposed about the elongate coupler shaft proximal of the microneedle 14 or microneedle array 14. A position sensor can be disposed about a proximal portion of the elongate coupler shaft and an electrical conductor electrically couples a the processor to the position sensor. The elongate coupler shaft driven by the driver coil pack controlled by the position sensor and the processor form the controllable driver, specifically, a controllable electromagnetic driver.

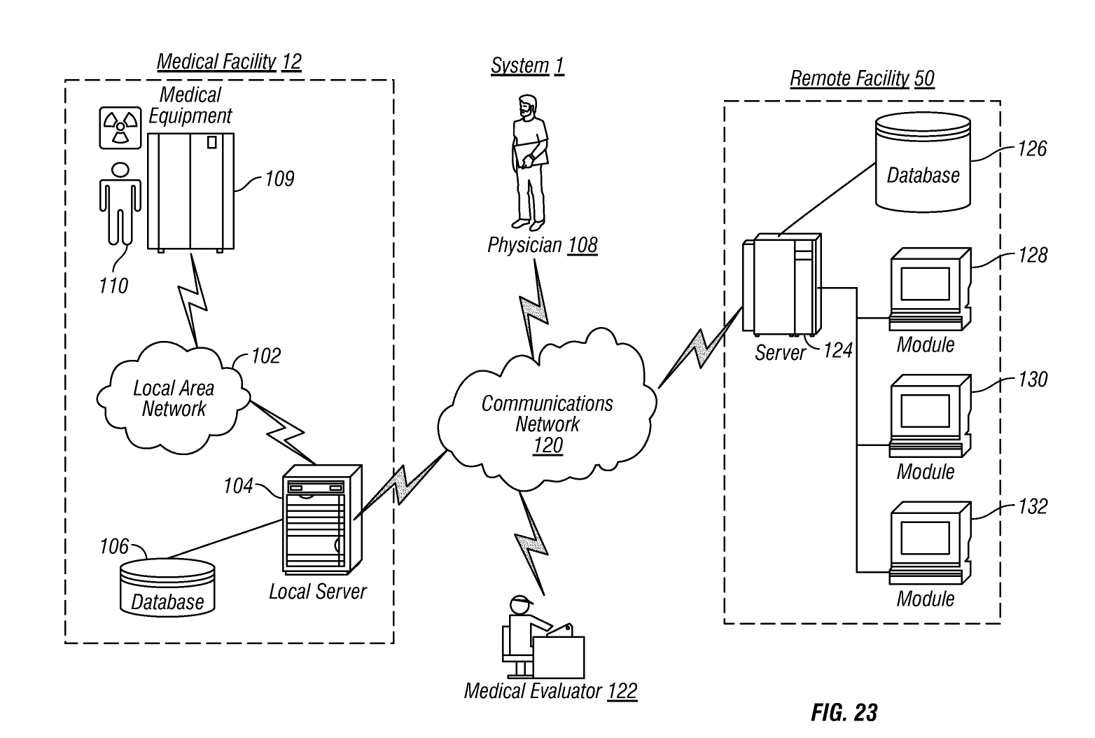

FIG. 23 shows an exemplary embodiment according to the present invention of a system 1 for providing remote analysis of medical data 102 of a patient 110. The medical data 102 from the device. The medical data 102 may be collected/generated at a medical facility 12 and transmitted, via a communications network 20, to a remote facility 50 for analysis.

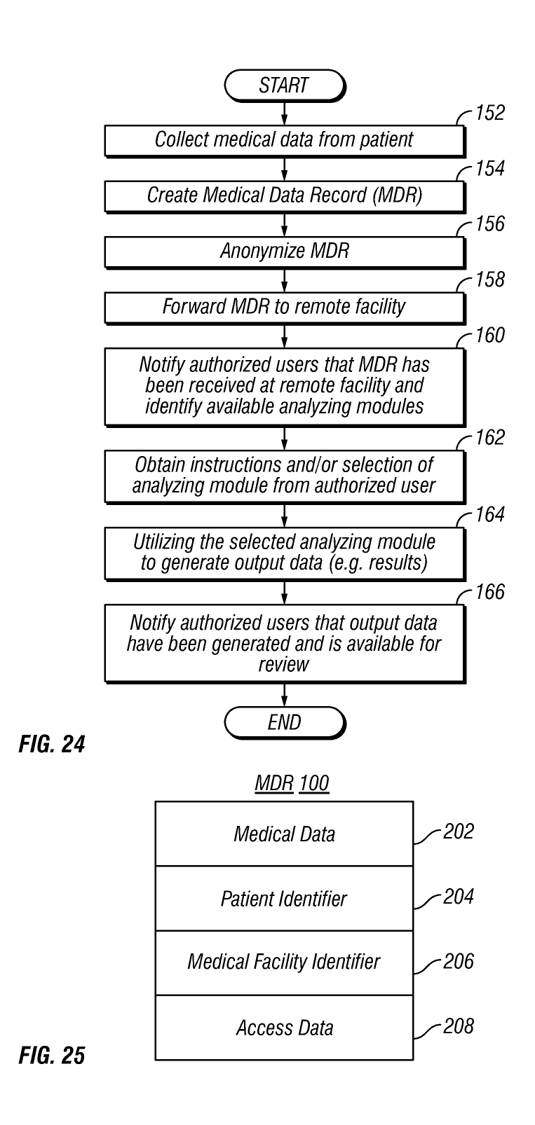

FIG. 24 shows an exemplary embodiment of the method according to the present invention. In step 152, the medical facility 12 collects the medical data 102 from the patient 110. In particular, the medical facility 12 may perform a medical procedure or analysis on the patient 10 using a medical device 109 to generate the medical data 102.

In step 154, the medical data 102 is forwarded to a local server 4, via a local area network 102, for creation of a Medical Data Record (“MDR”) 100. In particular, the MDR 100 is generated by the local server 104 using the medical data 102 along with other data which is described below.

FIG. 25 shows an exemplary embodiment of the MDR 100. The MDR 100 may include, in addition to the medical data 202, a patient identifier 204, a medical facility identifier 106 and an access data 208 indicating access parameters for the medical data 102. The patient identifier 204 may include patient's personal information (e.g., name, address, social security number, etc.). The access data 108 provides data regarding varying degrees of access to the MDR 100. For example, the access data 208 includes a list of authorized users and corresponding level of access. As would be understood by those skilled in the art, the authorized user may include a medical evaluator 22 (e.g., a radiologist), a physician 8, and/or other user functionaries.

In step 156, the MDR 100 is modified in preparation for transmission to the remote facility 50. In particular, the local server 104, to preserve patient's confidentiality and comply with HIPAA requirements, modifies the patient's identifier 104. In one exemplary embodiment, the local server 104 may assign a randomly generated anonymous identifier. Then, the patient's personal information (e.g., name, address, social security number, etc.) is removed from the patient's identifier 104 and replaced with the anonymous identifier. The local server 104 may store the patient's personal information along with the corresponding anonymous identifier in the database 106. Once corresponding output data is received from the remote facility 50, the local server 104 is able to determine the corresponding patient's personal information using the anonymous identifier.

In step 158, the medical facility 12 forwards the modified MDR 100 to the remote facility 50 via the communications network 20 (e.g., the Internet, a Wide Area Network or another computer communications network). The remote facility 50 may be external and independent of the medical facility 12 and located anywhere in the world.

The remote facility 50 may include a server 124, a database 126 which stores the MDR 100 and a plurality of analyzing modules 128, 130, 132, etc. The remote facility 50 is generally separate and independent form the medical facility 12. The remote facility 50 is responsible for obtaining (e.g., purchasing, leasing, etc.) and maintaining the analyzing modules 128-132. Each of the analyzing modules 128-132 may perform a designated task of analyzing the medical data 102. Thus, the analyzing module 128-132 receives as input the medical data 102, analyzes the medical data 102 and generates the output data.

The analyzing module 128-132 may include, for example, computer algorithms that utilize high-resolution data more efficiently to improve performance. The analyzing modules 128-132 may also include a remote analysis of patient data.

In one exemplary embodiment, one or more modules may include a management system such as the ELCAP management system (EMS). The EMS is a web-based management tool which includes image storage and analysis components; it manages all aspects of patient scheduling, clinical information, transfer of images, and image interpretation. The EMS also includes the highest quality measuring tools available that allow for volumetric measurement of nodules. However, it will be understood that the invention is not so limited and that it provides a universal platform with capability to incorporate substantially any number or type of computer analysis modules as they become available.

In step 160, the medical facility 12 and/or the remote facility 50 may notify (e.g., phone, fax, email) predefined authorized users, as listed in the access data 108, that the MDR 100 has been transmitted to or received by the remote facility 50 and is available for further analysis. In addition, the remote facility 50 provides information to the authorized users regarding availability and functionality of the analyzing modules 128-132.

In step 162, the authorized users can access the remote facility 50, e.g., via the communications network 120, by providing an access code. The authorized user provides an indication to the remote server 124 as to which module (e.g., the analyzing module 130) is selected to utilize for analysis of the medical data 102.

In step 164, the remote server 124 instructs the selected analyzing module 130 to perform the analysis of the medical data 102. The analyzing module 130 generates output data which is stored in the database 126. For example, the medical facility may forward the MDR that contains CT scan images of a patient's lungs to the remote facility for detection and measurement of nodules for lung cancer diagnosis. Before performing any manual review of the images, a radiologist may access the remote facility and select a particular analyzing module. The module analyzes the images, generates reports, flags certain images or a particular nodule for the radiologist, etc. These results may assist the radiologist in reviewing and issuing of a report.

In step 166, the authorized users are notified that the output data had been generated and is available for access. Alternatively, or in addition, the output data is transmitted to the medical facility 12. The medical facility 12 then using the anonymous patient identifier, determines the patient's personal information and stores the output data in corresponding patient's record.

One of the advantages of the present invention is that the medical facility 12 or any authorized user does not have to purchase and maintain the analyzing modules. On other hand, the analyzing modules 128-32 are available for analyzes when needed. For example, the analyzing modules 128-32 may be utilized on a pay-per-use basis or any other payment model desired. For example, monthly payments for usage up to a threshold level with pay-per-use charges for use in excess of the threshold level. For the pay-per-usage model, each analysis of the medical data 102 results in a predefined charge directly attributable to the corresponding patient 10, medical facility 12, physician 108 or nurse 122 and the like and, therefore, billable thereto or to a corresponding medical insurance company, and the like.

In addition, once the medical data -102 and the results have been stored in the database 126, they may be held in the database 126 indefinitely to provide immediate access to all authorized users. For example, if the patient 110 is admitted by a further medical facility and a further medical procedure is performed, a physician at the further medical facility may access the data by contacting the remote facility 50 (e.g., also based on pay-per-access basis) to view the prior medical data and related results.

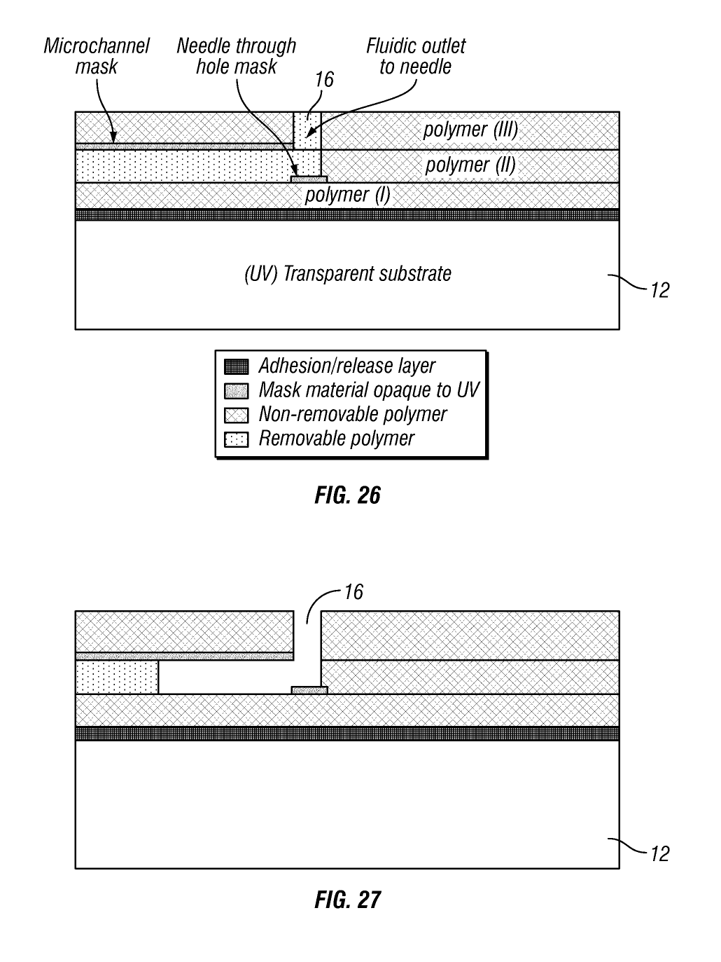

In one embodiment, monolithically formed polymeric microneedle 14 arrays with integrated microfluidics are created with the following method, as illustrated in FIGS. 26-34.

There are multiple choices of polymers that can be used in this invention. For simplicity, we use SU-8 as an example to demonstrate the process flow. Non-topological changes in the process, for example: dry etching, as opposed to backside exposure, of the polymer to create the needle taper, may be required when using other polymers:

As illustrated in FIG. 26, the microchannels 16 with multiple layers of polymer are outlets to the microneedles 14, generated by multiple layers of the polymer. FIG. 26. This is then followed by polymer development. It will be appreciated that partial development can be used at this point, see FIG. 27.

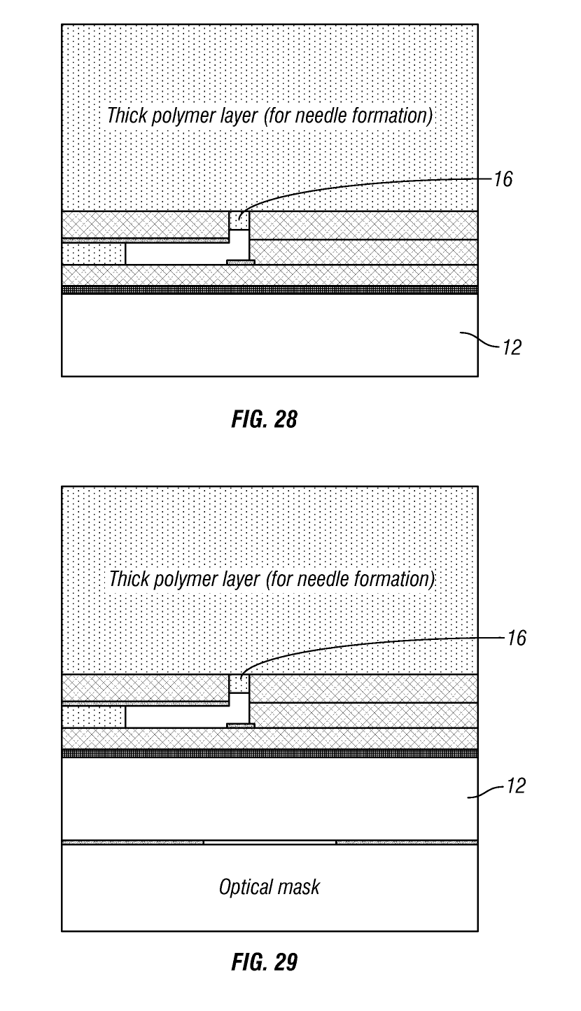

As illustrated in FIG. 28, a polymer layer is then deposited for microneedle 14 formation. Capillary force prevents spun-on polymer from entering the microchannels 16.

Contact lithography is used from the backside as shown in FIG. 29. A gap can be introduced between the mask and the sample for taper angle and microneedle 14 lateral dimension control. Exposure from top is possible via the use of external optical media (filters) that bend exposure beams.

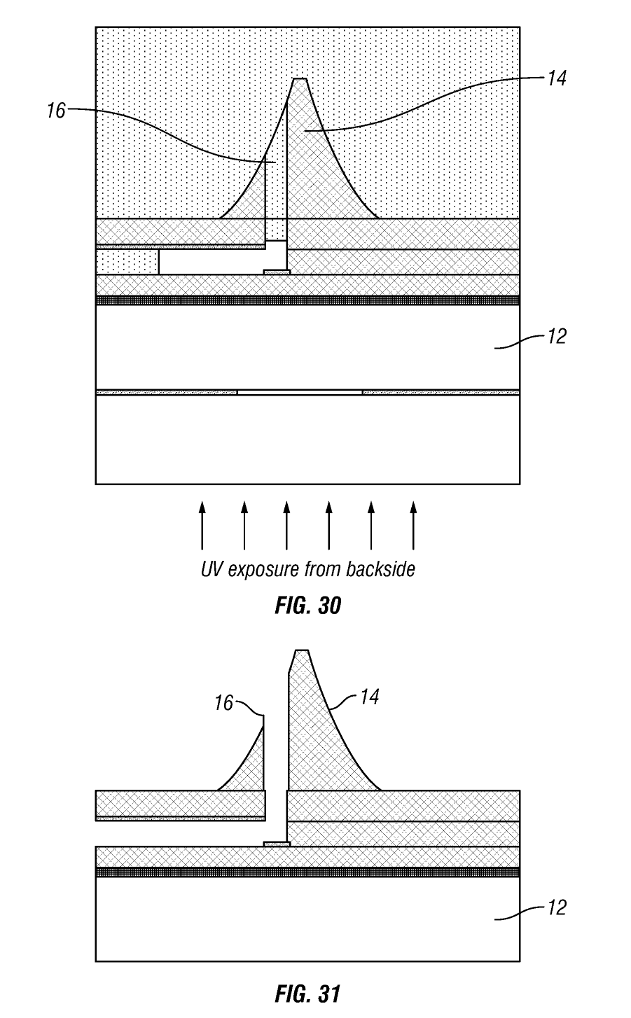

FIG. 30 illustrates microneedle 14 exposure. The degree of microneedle 14 taper depends on wavelength, dosage and exposure gap.

Polymer development is illustrated in FIG. 31, microneedle 14 structure is integrated with the microchannels 16 at this step.

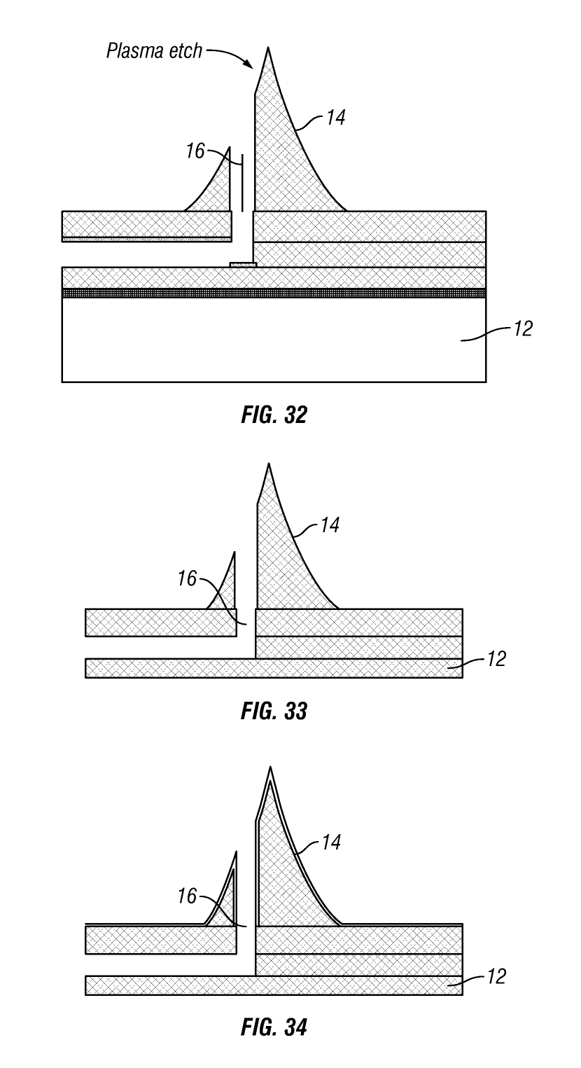

The microneedles 14 are then sharpened, see FIG. 32. In one embodiment, this is achieved by plasma sharpening. In one embodiment, SF₆/O₂ or CF₄/O₂ chemistry is used for the sharpening of polymeric microneedles 14. Other chemistries can be used including but not limited to Ar, and the like. Other polymers may require different dry etching chemistries, such as O2 and O2/Ar, and the like.

The device is then released as shown in FIG. 33. UV mask material can be removed after releasing device from a handle wafer.

Needle 14 surface treatments are then performed. These can include but are not limited to, (i) plasma surface roughening for enhanced metal adhesion, (ii) metal deposition for enhanced hardness and modulus, (iii) deposition of a material that covers the microneedle 14 surface and improves surface biocompatibility, including but not limited to parylene, and the like. Suitable metals provide, (i) a reasonable modulus, (ii) process compatibility to the underlying polymer, and (iii) that the metal inclusion does not jeopardize the overall biocompatibility of the system. Suitable metals include but are not limited to, tungsten, aluminum, and the like. Other materials can be used in place of a metal such as, silicon (semiconductor), deposited dielectrics, such as silicon oxide, or silicon nitride, and the like.

The final product is illustrated in FIG. 34.

The foregoing description of various embodiments of the claimed subject matter has been provided for the purposes of illustration and description. It is not intended to be exhaustive or to limit the claimed subject matter to the precise forms disclosed. Many modifications and variations will be apparent to the practitioner skilled in the art. Particularly, while the concept “component” is used in the embodiments of the systems and methods described above, it will be evident that such concept can be interchangeably used with equivalent concepts such as, class, method, type, interface, module, object model, and other suitable concepts. Embodiments were chosen and described in order to best describe the principles of the invention and its practical application, thereby enabling others skilled in the relevant art to understand the claimed subject matter, the various embodiments and with various modifications that are suited to the particular use contemplated.

What is claimed is:

1. A body fluid sampling or fluid delivery system, wherein the microchannels are capillary channels, comprising:
a polymeric support;
an array of polymeric microneedles coupled to the support, each of a microneedle, each of a microneedle having a height of 500 to 2000 μm and a tapering angle of 60 to 90°;
a plurality of polymeric microchannels each of a microchannel being associated with a microneedle, the plurality of polymeric microchannels being integrally formed with the array of polymeric microneedles without bonding; and
at least one polymeric reservoir coupled to the plurality of microchannels.

2. The system of claim 1, wherein the polymeric support is coupled to the array of polymeric microneedles without bonding.

3. The system of claim 1, wherein the plurality of polymeric microchannels and the array of microneedles are integrally formed to provide for controlled dimensions and alignment of the microchannels with the microneedles.

4. The system of claim 1, wherein the support, microneedles, microchannels and the reservoir are formed of the same polymer.

5. The system of claim 1, wherein analysis of a body fluid substance is in at least one of the microchannels and the reservoir.

6. The system of claim 1, wherein analysis of a body fluid stance is in the microchannels.

7. The system of claim 1, wherein a first reservoir is provided for incoming fluids, and a second reservoir is providing for outgoing fluids.

8. The system of claim 1, wherein the array of microchannels are capillary channels.

9. The system of claim 8, wherein the size of the reservoir is no greater than 1 uL.

10. The system of claim 8, wherein each of a microneedle has a distal end diameter of 50 to 100 μL.

11. The system of claim 8, wherein each of a microneedle has a size and geometry to provide for a fluid exit velocity of at least 100 m/second.

12. The system of claim 1, wherein number of microneedles coupled to the support is about 9 to 250.

13. The system of claim 1, wherein the microneedles are microjets.

14. The system of claim 1, wherein the polymer is SU-8.

15. The system of claim 1, wherein at least a portion of the microneedles can have an off-centered through hole for fluid transport.

16. The system of claim 1, wherein the microneedles have controlled taper to provide for improved tissue penetration with reduced limited material hardness.

17. The system of claim 1, wherein the microneedles include a deposited coating at an exterior surfaces for improved modulus and hardness.

18. The system of claim 17, wherein the deposited coating is selected from at least one of, a metal and a dielectric.

19. The system of claim 17, wherein the deposited coating has a thickness of 1-10 μm.

20. The system of claim 1, wherein each microneedle is tapered.

21. The system of claim 20, wherein each microneedle has a taper created by at least one of, (i) overexposure, (ii) near-field diffraction, (iii) mask distance adjustment and (iv) using external filters to change the incident angle of the UV.

22. The system of claim 1, wherein each microneedle is tapered and has a flexible structural topologies.

23. The system of claim 1, wherein the array of microneedles is formed on a wafer level.

24. The system of claim 1, wherein each microneedle is a hollow needle with a lumen sized to be small enough to draw only interstitial fluid and large enough to draw whole blood.

25. The system of claim 1, wherein each microneedle is not hollow and is dimensioned at a narrowest point of a tip to be 1 nm-300 um.

26. The system of claim 1, wherein each microneedle has a length of 2 um-2.0 cm.

27. The system of claim 1, wherein an outer diameter at a base opposite from a injection distal end of each microneedle is 20-gauge (1 mm) to 2 um.

28. The system of claim 1, wherein each microneedle has a lumen with a size of 1 um to 1 mm.

29. The system of claim 1, wherein each microchannels is 1 um to 3 mm

30. The system of claim 1, wherein each microneedle has an injector nozzle of 0.9 mm to 1 um.

31. The system of claim 30, wherein each injector nozzle injects 2 um to 2 cms.

32. The system of claim 1, wherein each microneedle has a geometry selected from at least one of, cylindrical, semi-cylindrical, conical, flat-sided, step pyramidal, a combination of different distal tip geometries, straight, diagonal and angled.

33. The system of claim 1, wherein the array of microneedles has geometric configurations to provide for spontaneous flow of a fluid through or past a distal end of the microneedles.

34. The system of claim 1, wherein each microneedle has a lumen that is offset for a longitudinal axis of the microneedle and not in a center of a distal end of the microneedle.

35. The system of claim 1, wherein capillary action is used to provide for body fluid flow through each microneedle.

36. The system of claim 1, wherein each microneedle includes a protective cap at a distal end of the microneedle.

37. The system of claim 1, further comprising:
a seal that is not in contact with distal ends of the array of microneedles.

38. The system of claim 1, further comprising:
a diaphragm to protect a sample of body fluid from ambient air.

39. The system of claim 1, wherein a distance between adjacent microneedles is 2 um to 2 cm.

40. The system of claim 1, wherein the array of microneedles has a total area (height×width) of 1 um to 4000 cm.

41. The system of claim 40, wherein the array of microneedles is a 24″×24″ array.

42. The system of claim 1, wherein each microneedle has a surface coating that interfaces with body tissue selected from at least one of, antimicrobial, anticoagulant, anti-stick agents, agents that have therapeutic effects on one or more body systems, diagnostic agents that include i.e. chemical substances used to reveal, pinpoint, and define localization of a pathological process, and genomic diagnostics.

43. The system of claim 42, wherein the surface coating extends from a distal end of each of a microneedle to about 2 um to 2 cm.

44. The system of claim 42, wherein the surface coating has a thickness of 1 angstrom to 10 um.

45. The system of claim 1, wherein the microneedles are configured to provide for body fluid withdrawal and injection of a fluid.

46. The system of claim 1, wherein the microneedles are sized for their distal ends of each microneedle to breach the skin, owning for skin surface tenting effects, and travel to a capillary bed but not extend to distal portions of nerve endings.

47. The system of claim 1, wherein controls are provided to control the introduction of the microneedles.

48. The system of claim 47, wherein the controls are selected from at least one of, velocity control, depth of penetration and braking.

49. The system of claim 1, wherein a depth of penetration of the microneedles through the skin and into a tissue site is 2 um to 2 cm.

50. The system of claim 1, wherein the array of microneedles has sufficient rigidity to be stiff enough to penetrate skin to a selected tissue site and sufficiently flexible to make a bend of a selected angle.

51. The system of claim 1, further comprising:
a device to assist in reducing an amount of pressure needed for skin penetration by the array of microneedles.

52. The system of claim 51, wherein the device to assist is selected from at least one of, vibration devices, electrical currents, and static or dynamic penetration.

53. The system of claim 1, wherein the capillary channels are coated or impregnated with different materials.

54. The system of claim 1, wherein the capillary channels are coated or impregnated with at least one a purified antibody selected from, CD3, CD4, CD4, CD7, CD8, CD15, CD19, CD20, CD34, CD45, CD57, Cytokeratin, HLA-DR, TCR (alpha beta), TCR (gamma delta), Bci-2, CD 16, CD1a, CD2, CD3 and CD4.

55. The system of claim 1, further comprising:
an electronic driver coupled to the array of microneedles.

56. A method of body fluid sampling from a patient, comprising:
providing a system with an array of microneedles and microchannels that are integrally formed;
introducing the array of microneedles into a patient;
collecting a body fluid from the patient in the sample chamber; and
measuring a parameter of the body fluid in the sample chamber.

57. The method of claim 56, wherein the parameter measured is used for blood typing.

58. The method of claim 56, wherein the parameter measured is used for performing diagnostic analysis (included but not limited to pharmacological testing, hematological analysis, body fluid analysis including but not limited to lymphatic fluid, interstitial fluid, urine, cerebrospinal fluid, intraocular fluids, biliary and ductal fluids, intra-cellular fluids; therapeutic treatments, delivery of pharmaceuticals, vaccinations, vitamins, minerals and therapeutic supplements; genomic diagnostics and gene removal; analysis of genetic diseases and disorders, stem cell removal, and genetic material removal, and the like; genetic therapies, delivery of stem cells, delivery of genetic materials into intraocular fluid and delivery of genetic materials into intracellular spaces.

59. The method of claim 56, wherein the collection occurs with a heel prick of the patient that can include a pinprick puncture made in a heel of a patient's foot.

60. The method of claim 56, wherein the parameter measured is used for O₂ analysis.

61. The method of claim 56, wherein no more than a volume of blood is collected of 0.01-1.0 milliliter.

62. The method of claim 56, wherein the patient is a neonate.

63. The method of claim 57, wherein the neonate is a low birth weight low birth weight of <1,500 gm, a very low birth weight of <1,000 gm and an extremely low birth weight of <500.

64. The method of claim 62, wherein blood gas concentration analysis of the neonate is performed at least every 6 hours on the neonate.

65. The method of claim 63, wherein the parameter measured is used to treat AOP.

66. The method of claim 56, wherein body fluids includes at least one of, blood from veins, venules, arteries, arterioles, capillaries, lymphatics, and interstitial fluid.

67. The method of claim 56 wherein body fluids include at least one of, urine, cerebrospinal fluid, intraocular fluids, biliary and ductal fluids.

68. The method of claim 56, wherein the microneedles are sufficiently small to provide for intra-cellular measurement.

69. The method of claim 56, wherein the parameter measured is used to at least treat a disease or condition that is, naturally occurring and caused by external means including but not limited to, radiation, bio-terrorism, oncological pollutants and poisons.
